(19) United States
(12) Patent Application Publication
(10) Pub. No.: US 2025/0205203 A1
(43) Pub. Date: Jun. 26, 2025

(54) SUBSTITUTED PYRAZOLYL COMPOUNDS AND METHODS OF USE THEREOF

(71) Applicant: Chinook Therapeutics Canada, Inc., Vancouver (CA)

(72) Inventors: Marc-Olivier Andrew Boily, Vancouver (CA); Jinyue Ding, Vancouver (CA); David Andrew Powell, Vancouver (CA); Juan Manuel Rueda Espinosa, Vancouver (CA); Tao Sheng, Vancouver (CA)

(21) Appl. No.: 17/925,166

(22) PCT Filed: May 17, 2021
(86) PCT No.: PCT/IB2021/054224

§ 371 Date: Nov. 14, 2022

Related U.S. Application Data
(60) Provisional application No. 63/026,373, filed on May 18, 2020.

(51) Int. Cl. A61K 31/427; A61K 31/36; A61K 31/422; A61K 31/4415; A61K 31/7105; A61K 35/74; A61K 38/51; C07D 417/04; C07D 417/14

(52) U.S. Cl. CPC A61K 31/427; A61K 31/36; A61K 31/422; A61K 31/4415; A61K 31/7105; A61K 35/74; A61K 38/51; C07D 417/04; C07D 417/14

(57) ABSTRACT

Provided herein are compounds, compositions and methods useful for inhibiting lactate dehydrogenase (LDH) activity and for the treatment, prevention and amelioration of one or more symptoms of diseases or disorders related to LDH activity, or the accumulation of oxalate, including hyperoxaluria.

### TECHNICAL FIELD

Provided herein are compounds, compositions and methods useful for inhibiting lactase dehydrogenase (LDH) activity and for the treatment, prevention and amelioration of one or more symptoms of hyperoxaluria, including primary hyperoxaluria and stone formation in the kidney and urinary tract.

### BACKGROUND

Hyperoxaluria is characterized by an increased concentration of oxalate in an individual, normally manifesting as elevated urinary oxalate excretion. Oxalate is a dicarboxylic acid which can form a complex with cations such as calcium to generate highly insoluble calcium oxalate crystals. Deposition of calcium oxalate crystals can impact kidney function, resulting in the formation of stones throughout the urinary tract (urolithiasis), kidneys (nephrolithiasis) and progressively increased levels of calcium in the kidneys (nephrocalcinosis) (National Organization for Rare Disorders—PH disease database). The overall implications are kidney damage, kidney stones, urinary tract-infections, chronic kidney disease and in some cases, end-stage-renal disease (ESRD). Furthermore, hyperoxaluria, combined with reduction in glomerular function can lead to systemic oxalosis, whereby oxalate depositions occurs throughout the body, including bones, retina, central-nervous tissue and the vasculature lining (Bhasin, World J. Nephrol. 2015, 42 (2), 235-244).

Hyperoxaluria is sub-divided into primary and secondary hyperoxalurias based on the clinical etiology. Primary hyperoxaluria (PH) is a genetic error of metabolism due to defective enzyme activity and is further divided into three subtypes (Harambat, Int. J. Nephrol. 2011:864580). Primary hyperoxaluria type I (PH1) is an autosomal recessive disorder caused by a deficiency of the liver-specific, peroxisomal enzyme, alanine-glyoxylate aminotransferase (AGT, gene name is AGXT). This is the most severe form of PH1, and accounts for approximately 80% of diagnosed PH cases. PH1 often develops during childhood or adolescence, and the disorder is characterized by recurrent kidney stones. Kidney failure is observed in approximately 20-50% of PH1 patients. The AGT enzyme is responsible for the detoxification of glyoxylate to glycine and competes with lactate dehydrogenase (LDH)-mediated conversion of glyoxylate to oxalate. Thus, loss of AGT function results in increased production of oxalate. The estimated prevalence of PH1 in Europe is 1-3 cases per million people and accounts for ˜1% of pediatric end-stage renal disease (ESRD) in registries from Europe, USA and Japan (Harambat 2011). Over 150 AGXT mutations have been identified in PH1, and this genetic diversity may explain the heterogeneous clinical manifestations and disease severity of PH1 patients. As such, there is a frequent delay in establishing the PH1 diagnosis, and the incidence rate may be under-represented (van der Hoeven, Nephrol. Dial. Transplant 2012, 27 (10), 3855-3862). Diagnosis of PH1 is made by either confirmed mutation in the AGXT gene or reduced AGT activity in a liver biopsy specimen (Williams, Hum. Mutat. 2009, 30, 910-917).

Primary hyperoxaluria type II (PH2) is caused by a deficiency in the glyoxylate reductase/hydroxypyruvate reductase (GRHPR) gene. The gene which encodes this enzyme is responsible for the conversion of glyoxylate to glycine, and mutations usually result in a loss of GRHPR function. PH2 is generally believed to have a milder clinical course than PH1 with a lower risk of ESRD, although nephrolithiasis and frequent kidney stones are common in these patients (Dhondup, Am. J. Transplant. 2018, 18, 253-257). Chronic as well as terminal renal insufficiency may occur in these patients (Kemper, Eur. J. Pediatr. 1997, 156 (7), 509-512).

Primary hyperoxaluria type III (PH3) is caused by mutations in the HOGA1 gene which encodes for the liver-specific, mitochondrial enzyme 4-hydroxy-2-oxoglutarate aldolase (Belostotsky, Am. J. Hum. Genet. 2010, 87 (3), 392-399). The exact role this enzyme plays in the production of oxalate is not fully understood, but a current hypothesis is that the substrate of HOGA1, 4-hydroxy-2-oxoglutarate (HOG), is capable of inhibiting GRHPR (Reidel, Biochim. Biophys. Acta. 2012, 1822 (10), 1544-1552). While cases of ESRD in PH3 patients are significantly less common than in those of PH1 patients, incidents of kidney stones remain high in this diseased population and the patient and hospitalization burden is significant. For example, approximately 50-65% of individuals with PH3 present with a stone prior to five years of age (Monico, Clin. J. Am. Soc. Nephrol. 2011, 6, 2289-2295), and while some individuals experience less kidney stone incidents during adolescence and their adult life, this is not true for all individuals. It has also been noted that the PH3 carrier frequency rate is 1:185, similar to that of PH1. The genetic prevalence is 1:136,000, making PH3 more common than originally thought based on clinical diagnosis (Hopp, J. Am. Soc. Nephrol. 2015, 26, 2559-2570).

Increased urinary oxalate concentrations in PH patients correlate with increased disease severity and progression to end-stage renal disease (Zhao, CJASN, 2016, 11 (1), 119-126). Clinically, urinary oxalate concentrations below <0.45 mmol/day are considered in the normal range. Patients with a urinary oxalate concentration of >2.4 mmol/day have a significantly higher risk of developing ESRD (Hazard Ratio=3.4). Higher urinary oxalate excretion at both diagnosis and follow-up are associated with poorer renal outcome in PH patients. On average, PH1 patients have higher urinary oxalate concentrations (average of 297 patients is 2.0 mmol/day) and not surprisingly renal survival was only 27% after 30 years of follow-up, as compared with 92% and 95% for PH2 and PH3 individuals, where the urinary oxalate concentrations are lower, but still well above the normal range. In addition, elevated 24-hour urinary oxalate excretion has also been associated with a higher risk of chronic kidney disease (CKD) progression and ESRD in individuals with CKD stages 2 to 4 (Waikar, JAMA Internal Medicine, 2019). These findings confirm the critical importance of urinary oxalate excretion as a predictor of renal survival and the potential therapeutic value of lowering urinary oxalate concentrations.

Secondary hyperoxaluria is a general term utilized for all other hyperoxalurias which are not caused by genetic factors. In enteric hyperoxaluria, the driver is thought to be enhanced absorption of dietary oxalate, or oxalate precursors, such as glycolate. These patients typically have a chronic underlying gastrointestinal disorder which results in malabsorption of oxalate, such as bariatric surgery complications, short bowel syndrome or Crohn's disease. Enteric hyperoxaluria occurs with a rate of 5-24% in patients with inflammatory bowel disease, ileal resection and modern bariatric surgery and thus represents a significant unmet medical need (Esker, Am. J. Nephrol. 2016, 44, 85-91). Diabetes mellitus and obesity are both independently associated with elevated urinary oxalate excretion, as well as increased risk of kidney stones, but it remains unclear whether hyperoxaluria in these individuals is caused by increased dietary absorption or endogenous synthesis of oxalate (reviewed in Efe, Curr. Opin. Nephrol. Hypertens. 2019). All other secondary hyperoxalurias, which are not enteric in nature are defined as idiopathic, meaning the etiology resulting in hyperoxaluria has not be ascertained.

There are currently no approved therapies which are generally effective for hyperoxaluria patients. Shortly after clinical evidence of increased oxalate excretion, patients are normally advised to increase their fluid intake (recommendation is >2 L/m² per day). Modification of diet to reduce dietary sources of oxalate and increase calcium supplementation to complex free oxalate in the GI tract and reduce oxalate absorption are also common but have limited value in PH patients. Pyridoxine (Vitamin B6) is a co-factor for AGT and has been found to be beneficial in lowering urinary oxalate by approximately 30% in PH1 patients but is ineffective for the majority of patients (Watts, Clin. Sci. Lond. 1985, 69, 87-90).

Dual liver and kidney transplantation is the only effective means of fully reversing hyperoxaluria in PH1 patients. The timing of the liver-kidney transplant is usually dictated by the stage of chronic kidney disease and time to ESRD of the patient (Cochat, Nephrol. Dial. Transplant. 2012, 27, 1729-1736). Liver-kidney transplantation should be planned pre-emptively before significant systemic oxalosis occurs. Conventional dialysis is not effective for the removal of plasma oxalate concentrations from hyperoxaluria patients but is often utilized in patients with stage 5 CKD while awaiting transplantation. In these patients, high efficacy dialysis is recommended, which can modestly slow the disease progression, but does not prevent ESRD (Ellis, Nephrol. Dial. Transplant. 2001, 16, 348-354).

Efforts to reduce the endogenous synthesis of oxalate should be effective in the treatment of hyperoxaluria. Lactate dehydrogenase A (LDHA) catalyzes the oxidation of glyoxylate to oxalate and is the final step in oxalate synthesis (Lluis, Biochim et Biophys Acta. 1977, 333-342). Therefore, inhibition of LDHA would reduce the oxidative conversion of glyoxylate to oxalate and represents a promising approach for the treatment of hyperoxaluric diseases. Indeed, knockdown of LDHA expression following siRNA treatment has been shown to reduce urinary oxalate in rodent PH1 models (Lai, Mol Ther. 2018, 26 (8): 1983-1995) and in early clinical studies with Nedosiran (PHYOX: A Safety and Tolerability Study of DCR-PHXC in Primary Hyperoxaluria Types 1 and 2, OHF 2019).

Liver-targeted LDH inhibition is expected to be well-tolerated. Humans with loss-of-function mutations in LDHA have been reported and have exercise-induced rhabdomyolysis (Miyajima, Muscle & Nerve. 1995, 18:874-878), however liver-targeted siRNA against LDHA did not cause exertional myopathy in mice (Lai, Mol Ther. 2018, 26 (8): 1983-1995) and treatment with Nedosiran has been well-tolerated in clinical trials (PHYOX: A Safety and Tolerability Study of DCR-PHXC in Primary Hyperoxaluria Types 1 and 2, OHF 2019). Furthermore, humans with LDHB loss-of-function mutations do not have any reported phenotype (Tanis, Am. J. Hum. Genet. 1977, 29:419-430).

Several LDH inhibitors have been described in the literature as oncolytic agents (Granchi, J. Med. Chem. 2011, 54, 1599-1612; Ward, J. Med. Chem. 2012, 55, 3285-3306; Kohlmann, J. Med. Chem. 2013, 56, 1023-1040; Fauber, Bioorg. Med. Chem. Lett. 2013, 20, 5533-5539; Purkey, ACS Med. Chem. Lett. 2016, 7, 896-901; Rai, J. Med. Chem. 2017, 60, 9184-9204). The majority of these inhibitors suffer from modest LDH inhibition, poor cellular penetration and poor pharmacokinetic properties, making their application as therapies challenging. Furthermore, the specific requirements of an effective agent for the treatment of hyperoxaluria differ significantly from those required for oncology. Most oncology agents have a systemic tissue distribution profile, ensuring the compound can inhibit cancer cells throughout the body. In addition, for oncology indications, compounds need to exhibit a high degree of cytotoxicity. In contrast, with hyperoxaluria, one desires a liver-targeted tissue distribution profile, with improved activity in hepatocytes and little or no cytotoxicity.

There remains a need for novel classes of liver-targeted small molecule therapeutics which can inhibit the LDH protein to be utilized for treatment of diseases such as primary hyperoxaluria and secondary hyperoxalurias, where reducing the amount of oxalate synthesis would be beneficial.

### SUMMARY

In certain embodiments, provided herein are compounds of Formula (I), or a pharmaceutically acceptable salt or solvate thereof. In certain embodiments, provided herein are compounds that are inhibitors of the lactate dehydrogenase (LDH) enzyme. In certain embodiments, the compounds as LDH inhibitors will confer therapeutic benefits associated with reducing oxalate levels, including lowering endogenous production of oxalate.

In certain embodiments, provided herein are compounds having the Formula (I), or a pharmaceutically acceptable salt or solvate thereof:

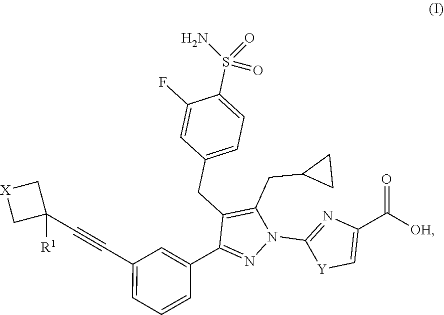

or a pharmaceutically acceptable salt or solvate thereof,
wherein X is —O— or —CH₂—;
Y is —O— or —S—; and
R¹ is hydrogen, hydroxyl, methyl or CF₃.

In certain embodiments, provided herein are compounds of Formula (I) selected from the group consisting of:

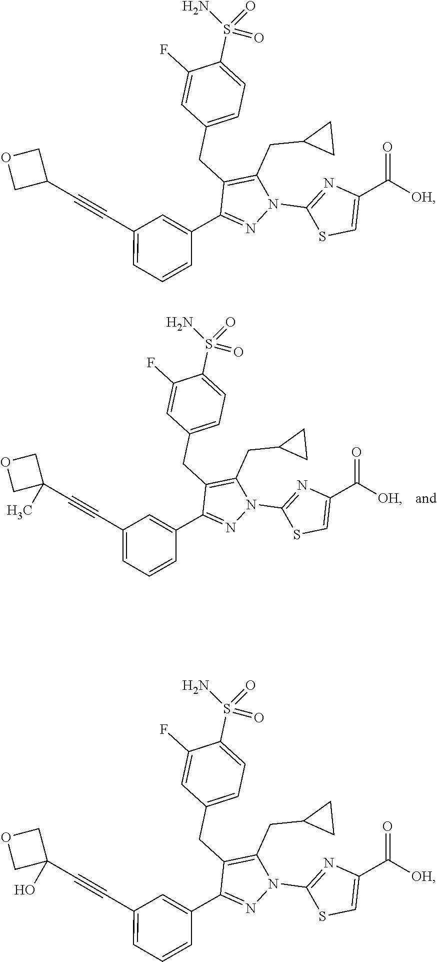 [SMILES: NS(=O)(=O)c1ccc(Cc2c(-c3cccc(C#CC4COC4)c3)nn(-c3nc(C(=O)O)cs3)c2CC2CC2)cc1F ; CC1(C#Cc2cccc(-c3nn(-c4nc(C(=O)O)cs4)c(CC4CC4)c3Cc3ccc(S(N)(=O)=O)c(F)c3)c2)COC1 ; "and" ; NS(=O)(=O)c1ccc(Cc2c(-c3cccc(C#CC4(O)COC4)c3)nn(-c3nc(C(=O)O)cs3)c2CC2CC2)cc1F]

or a pharmaceutically acceptable salt or solvate thereof.

In certain embodiments, provided herein is a compound selected from the group consisting of:

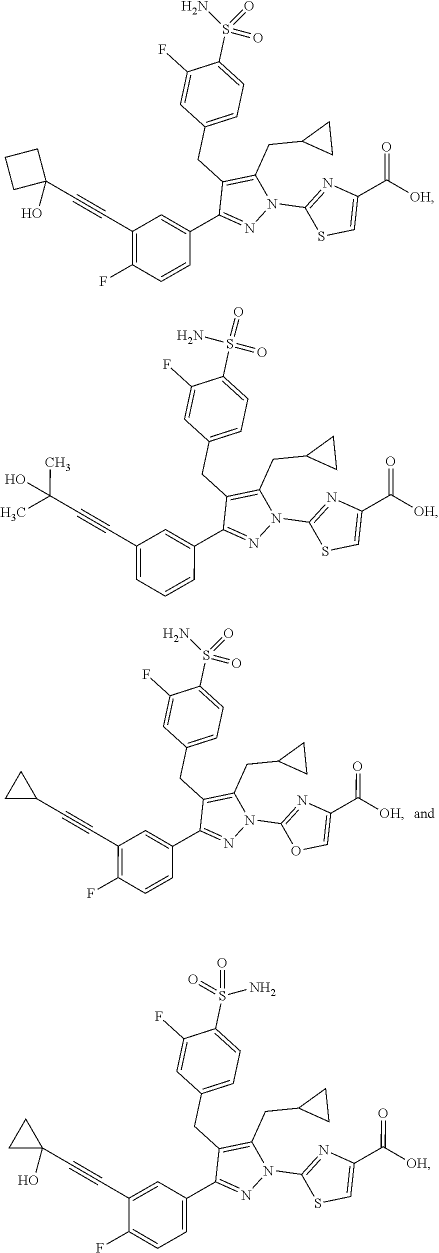

or a pharmaceutically acceptable salt or solvate thereof.

In certain embodiments, provided herein is a compound selected from the group consisting of:

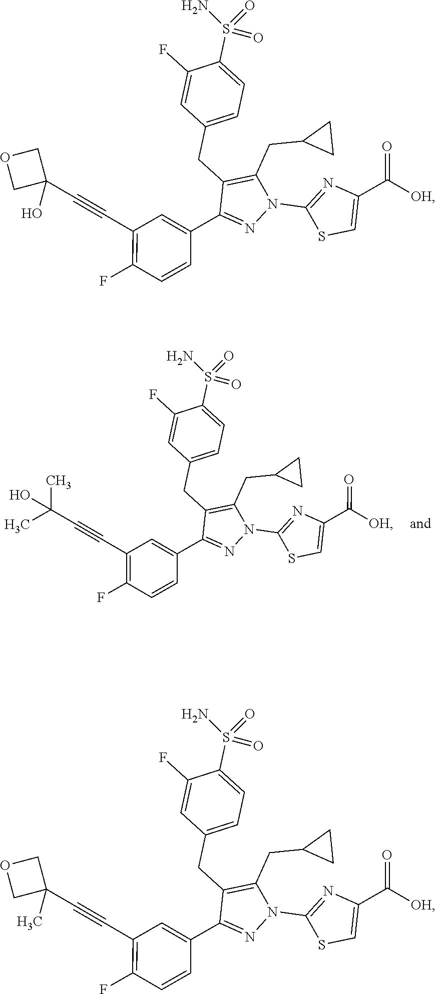 [SMILES: NS(=O)(=O)c1ccc(Cc2c(-c3ccc(F)c(C#CC4(O)COC4)c3)nn(-c3nc(C(=O)O)cs3)c2CC2CC2)cc1F ; CC(C)(O)C#Cc1cc(-c2nn(-c3nc(C(=O)O)cs3)c(CC3CC3)c2Cc2ccc(S(N)(=O)=O)c(F)c2)ccc1F ; "and" ; CC1(C#Cc2cc(-c3nn(-c4nc(C(=O)O)cs4)c(CC4CC4)c3Cc3ccc(S(N)(=O)=O)c(F)c3)ccc2F)COC1]

or a pharmaceutically acceptable salt or solvate thereof, for use treating a disease or disorder associated with elevated oxalate levels. In certain embodiments, the disease or disorder is hyperoxaluria, chronic kidney disease (CKD), end stage renal disease (ESRD) or kidney stone disease. In yet certain embodiments, the disease or disorder is primary hyperoxaluria, idiopathic hyperoxaluria or idiopathic oxalate kidney stone disease.

Also provided are pharmaceutical compositions formulated for administration by an appropriate route and means containing therapeutically effective concentrations of one or more of the compounds provided herein, or pharmaceutically acceptable salts or solvates thereof, and optionally comprising at least one pharmaceutical carrier. In certain embodiments, the pharmaceutical compositions deliver amounts effective for lowering oxalate levels in a subject in need thereof. In certain embodiments, the pharmaceutical compositions deliver amounts effective for reducing kidney stone formation in a subject in need thereof.

In another aspect, provided herein are methods of treating a disease or disorder associated with elevated oxalate levels, comprising administering to a subject having such disease or disorder, a therapeutically effective amount of one or more compounds disclosed herein, or a pharmaceutically acceptable salt or solvate thereof, or the pharmaceutical compositions disclosed herein. In certain embodiments, the disease or disorder is hyperoxaluria, chronic kidney disease (CKD), end stage renal disease (ESRD) or kidney stone disease. In yet certain embodiments, the disease or disorder is primary hyperoxaluria, idiopathic hyperoxaluria or idiopathic oxalate kidney stone disease. In yet certain embodiments, the disease or disorder is associated with the AGXT, GRHPR or HOGA1 mutation, or a combination of mutations thereof.

Also provided herein are combination therapies using one or more compounds or compositions provided herein, in combination with other pharmaceutical agents for the treatment of the diseases and disorders described herein.

These and other aspects of the subject matter described herein will become evident upon reference to the following detailed description and drawings.

### BRIEF DESCRIPTION OF THE DRAWING(S)

FIG. 1 depicts the percent reduction in urinary oxalate levels in AGXT knockdown mouse model of primary hyperoxaluria 1 (PH1) following administration of compounds disclosed herein, at 5 mg/kg QD PO for 5 days.

FIG. 2 depicts the XlogP distribution of the compounds disclosed herein.

### DETAILED DESCRIPTION

### A. Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise. The term “subject” refers to an animal which includes mammals such as mice, rats, cows, sheep, pigs, rabbits, goats, horses, monkeys, dogs, cats, and humans, including neonatal, infant, juvenile, adolescent, adult or geriatric patients.

The term “halo”, “halogen” or “halide” as used herein and unless otherwise indicated, refers to any radical of fluorine, chlorine, bromine or iodine.

The term “alkyl” as used herein and unless otherwise indicated, refers to a saturated hydrocarbon chain radical that may be a straight chain or branched chain, containing the indicated number of carbon atoms or otherwise having from one to ten, one to eight, one to six or one to four carbon atoms, and which is attached to the rest of the molecule by a single bond. In certain embodiments, the hydrocarbon chain is optionally deuterated. For example, C₁-C₆ alkyl indicates that the group may have from 1 to 6 (inclusive) carbon atoms in it. In some embodiments, an alkyl is a C₁-C₆ alkyl which represents a straight-chain or branched saturated hydrocarbon radical having 1 to 6 carbon atoms. Examples of alkyl include without limitation methyl, ethyl, n-propyl, isopropyl, n-butyl, isobutyl, sec-butyl, and tert-butyl.

The term “cycloalkyl” as used herein and unless otherwise indicated, refers to a monocyclic, bicyclic, tricyclic or other polycyclic hydrocarbon radical having the indicated number of ring carbon atoms or otherwise having three to ten carbon atoms and which are fully saturated or partially unsaturated. Multicyclic cycloalkyl may be fused, bridged or spiro-ring systems. Cycloalkyl groups include, but are not limited to, cyclopropyl, cyclobutyl, cyclopentyl, cyclohexyl, cycloheptyl, cyclooctyl, norbornyl, and partially unsaturated hydrocarbon rings such as cyclobutylene, cyclopentene and cyclohexene. In some embodiments, cycloalkyl is a monocyclic C₃-C₈ cycloalkyl.

The term “heteroaryl”, as used herein and unless otherwise indicated, represents a stable an aromatic 5-, 6- or 7-membered monocyclic- or stable 9- or 10-membered fused bicyclic ring system, which consists of carbon atoms and from one to four, or from one to three, heteroatoms selected from the group consisting of N, O and S wherein the nitrogen and sulfur heteroatoms may optionally be oxidized, and the nitrogen heteroatom may optionally be quaternized. In the case of a “heteroaryl” which is a bicyclic group, the second ring need not comprise a heteroatom and may be fused to a benzene ring. Accordingly, bicyclic “heteroaryl” includes, for example, a stable 5- or 6-membered monocyclic aromatic ring consisting of carbon atoms and from one to four, or from one to three, heteroatoms, as defined immediately above, fused to a benzene ring, or a second monocyclic “heteroaryl”, or a “heterocyclyl”, a “cycloalkyl”, or a “cycloalkenyl”, as defined above. Examples of heteroaryl groups include, but are not limited to, benzimidazole, benzopyrazole, benzisothiazole, benzisoxazole, benzofuran, isobenzofuran, benzothiazole, benzothiophene, benzotriazole, benzoxazole, furan, furazan, imidazole, indazole, indole, indolizine, isoquinoline, isothiazole, isoxazole, naphthyridine, oxadiazole, oxazole, phthalazine, pteridine, purine, pyrazine, pyrazole, pyridazine, pyridine, pyrimidine, pyrrole, quinazoline, quinoline, quinoxaline, tetrazole, thiadiazole, thiazole, thiophene, triazine, triazole, benzimidazole, benzothiadiazole, isoindole, pyrrolopyridines, imidazopyridines such as imidazo[1,2-a]pyridine, pyrazolopyridine, pyrrolopyrimidine and N-oxides thereof.

The term “hydrate” as used herein and unless otherwise indicated, refers to a compound provided herein or a salt thereof, that further includes a stoichiometric or non-stoichiometric amount of water bound by non-covalent intermolecular forces.

The term “in vivo” as used herein and unless otherwise indicated, refers to a process or event occurring in a living organism or living system.

The term “calcium oxalate stones” as used herein and unless otherwise indicated, refers to crystalline material comprising calcium oxalate salt present as stones or plaques in the kidney, bladder or urinary tract.

The term “hyperoxaluria” refers to a condition characterized by elevated levels of oxalate in the urine or plasma, or by the presence of kidney stones. In certain embodiments, hyperoxaluria is characterized by urinary oxalate excretion rate of greater than about 0.5 mmol/1.73 m² per day, greater than about 0.7 mmol/1.73 m² per day, greater than about 0.8 mmol/1.73 m², greater than about 1.0 mmol/7.2 m² per day, greater than about 1.2 mmol/1.73 m² per day or greater than about 2 mmol/1.73 m² per day. In certain embodiments, elevated oxalate levels means having an oxalate excretion rate that is greater than normal urinary excretion, which is less than about 0.45 mmol/1.73 m² per day. In certain embodiments, elevated oxalate levels means having a urinary oxalate excretion rate that is greater than about 40 mg/day. In certain embodiments, elevated oxalate levels means having a urinary oxalate excretion rate that is greater than about 45 mg/day. In certain embodiments, the urinary oxalate excretion rate is about two-fold higher than normal. In certain embodiments, the urinary oxalate excretion rate is about four-fold higher than normal. In yet certain embodiments, hyperoxaluria is characterized by urinary oxalate/creatinine ratio greater than the reference range for age. In certain embodiments, hyperoxaluria is characterized by glycolate/creatinine ratio greater than the reference range for age. Hyperoxaluria includes both primary hyperoxaluria and secondary hyperoxaluria.

“Primary hyperoxaluria” refers to a condition characterized by the overproduction of oxalate and/or defective production or function of one or more enzymes that regulate the levels of oxalate in the body. In certain embodiments, the primary hyperoxaluria is associated with deficiency in the expression of alanine: glyoxylate aminotransferase (AGT) or a mutation in AGXT, the gene encoding AGT, and may be classified as Type 1 primary hyperoxaluria, or PH1. In certain embodiments, the primary hyperoxaluria is associated with deficiency in the expression of glyoxylate reductase (GR) or a mutation in the gene encoding GR (GRPHR), and which may be classified as Type 2 primary hyperoxaluria, or PH2. In yet other embodiments, the primary hyperoxaluria is associated with the deficiency in the expression of 4-hydroxy-2-oxoglutarate aldolase (HOGA) or a mutation in the gene encoding HOGA (HOGA1), and which may be classified as Type 3 primary hyperoxaluria, or PH3.

“Secondary hyperoxaluria” refers to a condition characterized by elevated levels of oxalate in the urine or plasma, or the presence of kidney stones. Secondary hyperoxaluria includes enteric hyperoxaluria caused for example, by increased intake and intestinal absorption of dietary oxalate, excessive intake of oxalate precursors and alteration in the intestinal microflora. Secondary hyperoxaluria also includes idiopathic hyperoxaluria, of unknown etiology.

The term “solvate” as used herein and unless otherwise indicated, refers to a solvate formed from the association of one or more solvent molecules to a compound provided herein. The term “solvate” includes hydrates (e.g., mono-hydrate, dehydrate, trihydrate, and the like).

The term “treating”, “treat”, or “treatment” refers generally to administering one or more pharmaceutical substances, especially at least one compound of Formula (I) to a patient that has a disease, disorder or condition, or has a symptom or condition of a disease or disorder, or has a predisposition toward a disease or disorder, with the purpose to cure, heal, relieve, alter, alleviate, ameliorate, slow the progress of, delay the onset of, reduce the risk of, improve or affect the disease, disorder or condition or one or more symptoms thereof or the predisposition toward the disease, disorder or condition or its recurrence.

The term “therapeutically effective amount” or “effective amount” is an amount sufficient to effect beneficial or desired clinical results. An effective amount can be administered in one or more administrations. An effective amount is typically sufficient to palliate, ameliorate, stabilize, reverse, slow or delay the progression of the disease state or to treat the disease or condition.

In the description herein, if there is any discrepancy between a chemical name and chemical structure, the chemical structure controls.

### B. Compounds

In certain embodiments, provided herein are compounds having the Formula (I):

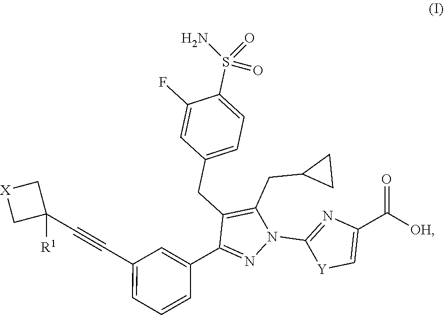

or a pharmaceutically acceptable salt or solvate thereof, wherein:
X is —O— or —CH₂—;
Y is —O— or —S—; and
R¹ is hydrogen, hydroxyl, methyl or CF₃.

In certain embodiments, provided herein are compounds having the Formula (I) wherein X is —O—. In certain embodiments, provided herein are compounds having the Formula (I) wherein Y is —S— and the other variables are as described for Formula (I) elsewhere herein. In certain embodiments, provided herein are compounds having the Formula (I) wherein R¹ is hydrogen, hydroxyl or methyl and the other variables are as described for Formula (I) elsewhere herein.

In certain embodiments, provided herein is a compound of Formula (I) wherein the compound is selected from the group consisting of:

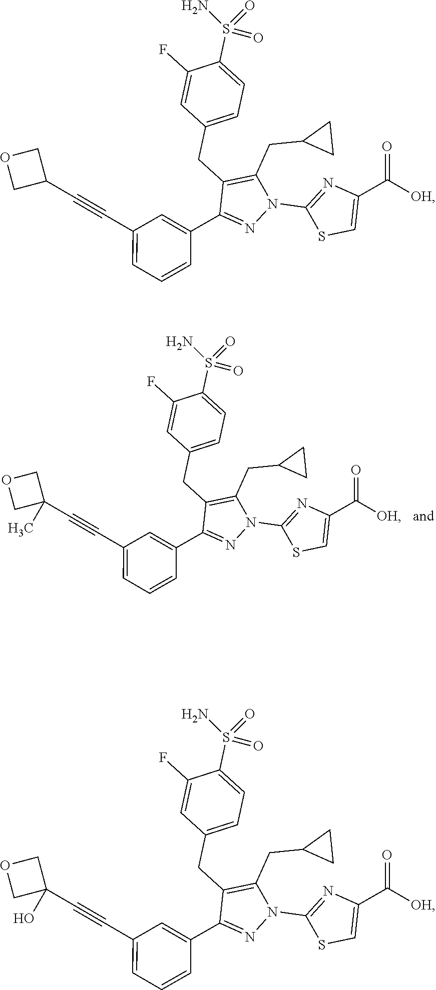 [SMILES: NS(=O)(=O)c1ccc(Cc2c(-c3cccc(C#CC4COC4)c3)nn(-c3nc(C(=O)O)cs3)c2CC2CC2)cc1F ; CC1(C#Cc2cccc(-c3nn(-c4nc(C(=O)O)cs4)c(CC4CC4)c3Cc3ccc(S(N)(=O)=O)c(F)c3)c2)COC1 ; "and" ; NS(=O)(=O)c1ccc(Cc2c(-c3cccc(C#CC4(O)COC4)c3)nn(-c3nc(C(=O)O)cs3)c2CC2CC2)cc1F]

or a pharmaceutically acceptable salt or solvate thereof.

In certain embodiments, provided herein is a compound selected from the group consisting of:

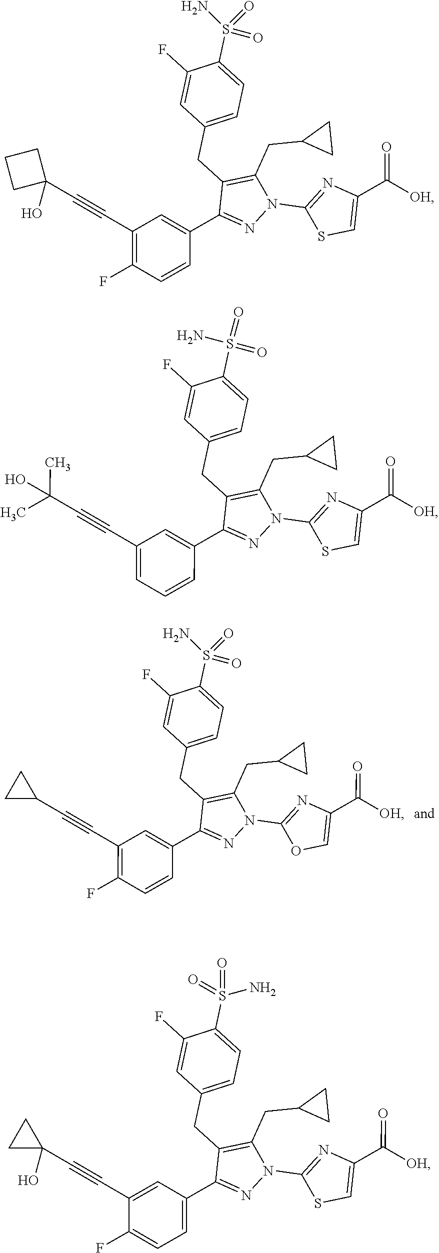

or a pharmaceutically acceptable salt or solvate thereof.

In certain embodiments, provided herein is a compound selected from the group consisting of:

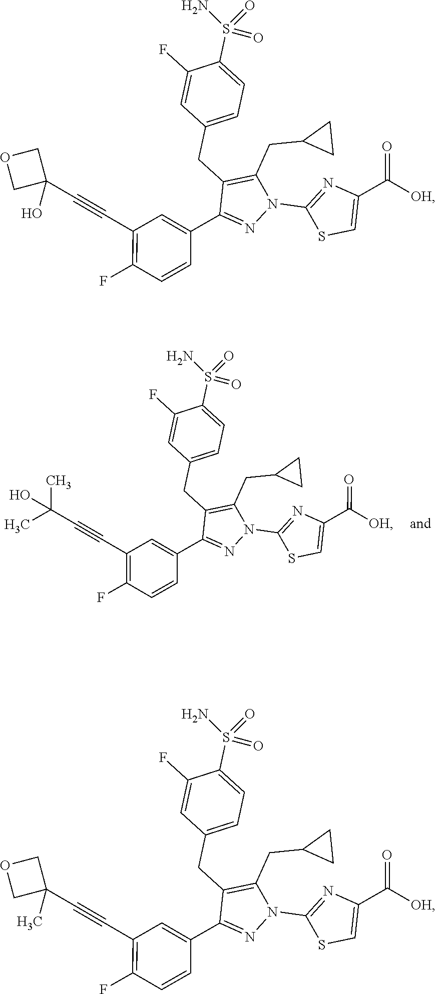 [SMILES: NS(=O)(=O)c1ccc(Cc2c(-c3ccc(F)c(C#CC4(O)COC4)c3)nn(-c3nc(C(=O)O)cs3)c2CC2CC2)cc1F ; CC(C)(O)C#Cc1cc(-c2nn(-c3nc(C(=O)O)cs3)c(CC3CC3)c2Cc2ccc(S(N)(=O)=O)c(F)c2)ccc1F ; "and" ; CC1(C#Cc2cc(-c3nn(-c4nc(C(=O)O)cs4)c(CC4CC4)c3Cc3ccc(S(N)(=O)=O)c(F)c3)ccc2F)COC1]

or a pharmaceutically acceptable salt or solvate thereof, for use in treating a disease or disorder associated with elevated oxalate levels. In certain embodiments, the disease or disorder is hyperoxaluria. In yet certain embodiments, the disease or disorder is primary hyperoxaluria, idiopathic hyperoxaluria or idiopathic oxalate kidney stone disease.

In certain embodiments, provided herein are pharmaceutical composition comprising a compound disclosed herein and a pharmaceutically acceptable carrier.

In certain embodiments, provided herein are isotopically enriched analogs of the compounds disclosed herein, for example, deuterated analogs, to improve pharmacokinetics (PK), pharmacodynamics (PD) and toxicity profiles of the compounds.

Any combination of the groups described above for the various variables is contemplated herein. Throughout the specification, groups and substituents thereof are chosen by one skilled in the field to provide stable moieties and compounds.

The compounds of the present disclosure include the compounds themselves, as well as their salts, solvate and solvate of the salt, if applicable. Salts for the purposes of the present disclosure are preferably pharmaceutically acceptable salts of the compounds according to the present disclosure. Salts which are not themselves suitable for pharmaceutical uses but can be used, for example, for isolation or purification of the compounds according to the disclosure are also included. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion.

As used herein, “pharmaceutically acceptable salts” refer to acid or base addition salts, including but not limited to, base addition salts formed by the compound of Formula (I) having an acidic moiety with pharmaceutically acceptable cations, for example, sodium, potassium, magnesium, calcium, aluminum, lithium, and ammonium.

Lists of suitable salts may be found in Remington's Pharmaceutical Sciences, 17th ed., Mack Publishing Company, Easton, Pa., 1985, p. 1418; S. M. Berge et al., “Pharmaceutical Salts”, J. Pharm. Sci. 1977, 66, 1-19; and “Pharmaceutical Salts: Properties, Selection, and Use. A Handbook”; Wermuth, C. G. and Stahl, P. H. (eds.) Verlag Helvetica Chimica Acta, Zurich, 2002 [ISBN 3-906390-26-8]; each of which is incorporated herein by reference in its entirety.

Solvates in the context of the present disclosure are designated as those forms of the compounds according to the present disclosure which form a complex in the solid or liquid state by stoichiometric coordination with solvent molecules. Hydrates are a specific form of solvates, in which the coordination takes place with water. The formation of solvates is described in greater detail in “Solvents and Solvent Effects in Organic Chemistry”; Reichardt, C. and Welton T.; John Wiley & Sons, 2011 [ISBN: 978-3-527-32473-6], the contents of which is incorporated herein by reference in its entirety.

In some embodiments, the compound of Formula (I) is present in pharmaceutically acceptable salt form. In some embodiments, the compound of Formula (I) is present in free acid form. In some embodiments, the compound of Formula (I) is present in free acid form. In some embodiments, the compound of Formula (I) is present in the form of a solvate. In some embodiments, the solvate is a hydrate. In some embodiments, the compound of Formula (I) is present as a solvate of a pharmaceutically acceptable salt form.

The present disclosure also encompasses all suitable isotopic variants of the compounds according to the present disclosure, whether radioactive or not. An isotopic variant of a compound according to the present disclosure is understood to mean a compound in which at least one atom within the compound according to the present disclosure has been exchanged for another atom of the same atomic number, but with a different atomic mass than the atomic mass which usually or predominantly occurs in nature. Examples of isotopes which can be incorporated into a compound according to the present disclosure are those of hydrogen, carbon, nitrogen, oxygen, fluorine, chlorine, bromine and iodine, such as ²H (deuterium), ³H (tritium), ¹³C, ¹⁴C, ¹⁵N, ¹⁷O, ¹⁸O, ¹⁸F, ³⁶Cl, ⁸²Br, ¹²³I, ¹²⁴I, ¹²⁵I, ¹²⁹I and ¹³¹I. Particular isotopic variants of a compound according to the present disclosure, especially those in which one or more radioactive isotopes have been incorporated, may be beneficial, for example, for the examination of the mechanism of action or of the active compound distribution in the body. Compounds labelled with ³H, ¹⁴C and/or ¹⁸F isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required. In some embodiments, hydrogen atoms of the compounds described herein may be replaced with deuterium atoms. In certain embodiments, “deuterated” as applied to a chemical group and unless otherwise indicated, refers to a chemical group that is isotopically enriched with deuterium in an amount substantially greater than its natural abundance. Isotopic variants of the compounds according to the present disclosure can be prepared by various, including, for example, the methods described below and in the working examples, by using corresponding isotopic modifications of the particular reagents and/or starting compounds therein.

In certain embodiments, the compounds provided herein have physicochemical properties which promote a liver targeted tissue distribution profile, and which maximizes their exposure in the liver while minimizing exposure in other tissues (e.g. plasma, muscle, spleen, testes). In certain embodiments, the compounds provided herein have demonstrated a liver-targeted tissue distribution profile in rodents, as evident by a greater drug exposure in liver versus other tissues (e.g. plasma, muscle), for example, 4 h after PO dosing or 24 h after PO dosing. In certain embodiments, the compounds provided herein have physicochemical properties that promote uptake by OATP subfamily of receptors that are expressed in the liver. The OATP subfamily members OATP1B1, OATP1B3 and OATP2B1 are transporters principally expressed on human hepatocytes, where they mediate the uptake of substrates from blood to liver. Hence, OATP substrates are expected to have higher exposure in the liver compared to systemic and peripheral tissues. In certain embodiments, the compounds provided herein are substrates for OATP. In certain embodiments, the compounds provided herein are substrates for OATP1B1, OATP1B3, OATP2B1 or a combination thereof. In yet certain embodiments, the compounds provided herein have physicochemical properties that minimize passive diffusion of the compounds into off-target tissues lacking membrane transporters, thereby promoting liver selectivity. In certain embodiments, the compounds provided herein have octanol-water partition coefficients (XlogP) that fall in a range that minimizes their passive diffusion potential, promotes liver targeting and reduces exposure to tissues outside the liver. In certain embodiments, the compounds provided herein have a calculated octanol-water partition coefficient (XlogP) of about 4 to about 6.5. In certain embodiments, the compounds provided herein have an XlogP of about 4.5 to about 6. In yet certain embodiments, the compounds provided herein have an XlogP of about 5.5 to about 6. In yet certain embodiments, the compounds provided herein have an XlogP of about 4 to about 5.5. In yet certain embodiments, the compounds provided herein have an XlogP of about 4 to about 5.

### C. Formulation

The term “pharmaceutical composition” as used herein is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt, or solvate or solvate of the salt thereof, and a pharmaceutically acceptable carrier.

The term “pharmaceutically acceptable carrier” refers to a carrier or an adjuvant that may be administered to a patient, together with a compound of the present disclosure, or a pharmaceutically acceptable salt, solvate, salt of the solvate or prodrug thereof, and which does not destroy the pharmacological activity thereof and is nontoxic when administered in doses sufficient to deliver a therapeutic amount of the compound.

The amount administered depends on the compound formulation, route of administration, etc. and is generally empirically determined, and variations will necessarily occur depending on the target, the host, and the route of administration, etc. Generally, the quantity of active compound in a unit dose of preparation may be varied or adjusted from about 1 milligram (mg) to about 100 mg or from about 1 mg to about 1000 mg, according to the particular application. For convenience, the total daily dosage may be divided and administered in portions during the day.

Solid dosage forms of the instant pharmaceutical compositions for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid pharmaceutical compositions of a similar type may also be employed as fillers in soft and hard-filled gelatin capsules using such excipients as lactose or milk sugar as well as high molecular weight polyethylene glycols and the like.

The solid dosage forms of the instant pharmaceutical compositions of tablets, dragées, capsules, pills, and granules can be prepared with coatings and shells such as enteric coatings and other pharmaceutical coatings. They may optionally contain opacifying agents and can also be of a formulation that they release the active ingredient(s) only, or preferentially, in a certain part of the intestinal tract, optionally, in a delayed manner. Examples of embedding pharmaceutical compositions which can be used include polymeric substances and waxes.

The active compounds can also be in microencapsulated form, if appropriate, with one or more of the above-mentioned excipients.

Liquid dosage forms of the instant pharmaceutical compositions for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions of the instant compounds, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Pharmaceutical compositions of the present disclosure for injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The pharmaceutical compositions that are injectable formulations can be sterilized, for example, by filtration through a bacterial-retaining filter, or by incorporating sterilizing agents in the form of sterile solid pharmaceutical compositions that can be dissolved or dispersed in sterile water or other sterile injectable medium prior to use.

Besides inert diluents, these pharmaceutical compositions may also contain adjuvants such as preservative, wetting agents, emulsifying agents, suspending agents, dispersing agents, sweetening, flavoring, and perfuming agents.

Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminum monostearate and gelatin. The compounds can be incorporated into slow release or targeted delivery systems such as polymer matrices, liposomes, and microspheres. Such formulations may provide more effective distribution of the compounds.

Dosage forms for topical administration of a compound or pharmaceutical composition of the present disclosure include powders, patches, sprays, ointments and inhalants. The active compound is mixed under sterile conditions with a pharmaceutically acceptable carrier and any preservatives, buffers, or propellants which may be required.

The compounds and compositions described herein can, for example, be administered orally, parenterally (e.g., subcutaneously, intracutaneously, intravenously or intramuscularly), topically, rectally, nasally sublingually or buccally, with a dosage ranging from about 0.01 milligrams per kilogram (mg/kg) to about 1000 mg/kg, (e.g., from about 0.01 to about 100 mg/kg, from about 0.1 to about 100 mg/kg) every 4 to 120 hours, or according to the requirements of the particular drug, dosage form, and/or route of administration. Other routes of administration include enteric, intraarterial, intraperitoneal and intrathecal administration. The interrelationship of dosages for animals and humans (based on milligrams per meter squared of body surface) is described by Freireich et al., Cancer Chemother. Rep. 50, 219-244 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or by injection. The methods herein contemplate administration of an effective amount of compound or compound composition to achieve a desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, and the judgment of the treating physician.

Dosage forms include from about 0.001 mg to about 2,000 mg (including, from about 0.001 mg to about 1,000 mg, from about 0.001 mg to about 500 mg, from about 0.01 mg to about 250 mg) of a compound of Formula (I), or a salt (e.g., a pharmaceutically acceptable salt) thereof as defined anywhere herein. The dosage forms can further include a pharmaceutically acceptable carrier and/or an additional therapeutic agent.

Appropriate dosage levels may be determined by any suitable method. Preferably, the active substance is administered at a frequency of 1 to 4 times per day for topical administration, or less often if a drug delivery system is used. Nevertheless, actual dosage levels and time course of administration of the active ingredients in the pharmaceutical compositions of the present disclosure may be varied so as to obtain an amount of the active ingredient which is effective to achieve a desired therapeutic response for a particular patient, composition and mode of administration, without being intolerably toxic to the patient. In certain cases, dosages may deviate from the stated amounts, in particular as a function of age, gender, body weight, diet and general health status of the patient, route of administration, individual response to the active ingredient, nature of the preparation, and time or interval over which administration takes place. Thus, it may be satisfactory in some cases to manage with less than the aforementioned minimum amount, whereas in other cases the stated upper limit may be exceeded. It may in the event of administration of larger amounts be advisable to divide these into multiple individual doses spread over the day.

### D. Evaluation of the Activity of the Compounds

Standard physiological, pharmacological and biochemical procedures are available for testing the compounds to identify those that possess biological activity as LDH inhibitors.

Biochemical assays include recombinant human LDH enzymatic assays in which purified recombinant human lactate dehydrogenase A (LDHA) is incubated with test compound, substrate pyruvate and coenzyme NADH+, and its enzymatic activity measured by the formation of NAD upon conversion of pyruvate to lactate.

LDH inhibitors can also be evaluated in an ex vivo assay consisting of primary mouse hepatocytes. Following isolation, viable wild-type murine hepatocytes are incubated with test compound in presence of pyruvate. Compound potency to modulate LDH enzymatic activity is then evaluated by measuring the conversion of pyruvate to lactate by the cells.

Genetically engineered alanine-glyoxylate aminotransferase-deficient mice such as knockout AGT^{-/-} may also serve as a primary hyperoxaluria model. In addition, silencing of AGT hepatic expression can be rendered via sustained liver-targeted RNA interference in both wild-type rats and mice. In some specific cases, model may also require saturation of the glycolate metabolic pathway through chronic exposure to ethylene glycol or sodium glycolate. In all instance, the efficacy of test compounds is assessed by their potency to reduce the urinary oxalate or glycolate burden [primary endpoint], which is expressed either as oxalate/creatinine ratio, or as the total amount of oxalate excreted over a 24-hour period. Additional endpoints can be considered, including histological evaluation of structural integrity of kidneys and presence of calcium oxalate crystal deposition, as well as renal function assessment (e.g. estimated glomerular filtration rate or eGFR).

### E. Methods of Use

LDH inhibitors may prove to be effective for diseases resulting from an increase in oxalate or where oxalate reduction may be beneficial. An example is primary hyperoxaluria, which is a disease resulting from an overproduction of oxalate, for example, due to overproduction or accumulation of its precursor, glyoxylate. Provided herein therefore are methods of treating or preventing diseases or disorders associated with elevated oxalate levels. Diseases or disorders associated with elevated oxalate levels include hyperoxaluria, chronic kidney disease (CKD), end stage renal disease (ESRD) or kidney stone disease. In certain embodiments, the hyperoxaluria is associated with various digestive or bowel diseases such as Crohn's diseases, Hirschspring's disease, cystic fibrosis and chronic biliary or pancreatic pathology. In certain embodiments, the hyperoxaluria is associated with bariatric surgery and ileal resection. In certain embodiments, the chronic kidney disease is associated with diabetes, hypertension, previous episode(s) of acute kidney injury, cardiovascular disease or dyslipidemia. In certain embodiments, the kidney stone disease is idiopathic kidney stone disease, or kidney stone disease associated with hyperparathyroidism or other disorders of calcium metabolism. In certain embodiments, the elevated oxalate levels is associated with diabetes mellitus, obesity or metabolic syndrome (MS). The compounds and compositions provided herein may be used to treat or prevent hyperoxaluria, including primary hyperoxaluria and the subtypes PH1, PH2 and PH3 as well as secondary hyperoxaluria, including enteric hyperoxaluria and idiopathic hyperoxaluria. The compounds and compositions provided herein may be used to treat calcium oxalate stone formation, for example, in the kidney, urinary tract or bladder, treat calcium oxalate deposition in other tissues and organs outside the kidney (systemic oxalosis) or prevent or delay kidney damage or the onset of chronic kidney disease (CKD) or end stage renal disease (ESRD).

In certain embodiments, elevated oxalate levels means having a urinary oxalate excretion rate of greater than about 0.5 mmol/1.73 m² per day, greater than about 0.7 mmol/1.73 m² per day, greater than about 0.8 mmol/1.73 m² per day, greater than about 1.0 mmol/1.73 m² per day, greater than about 1.2 mmol/1.73 m² per day or greater than about 2 mmol/1.73 m² per day. In certain embodiments, elevated oxalate levels means having a urinary oxalate excretion rate that is greater than normal urinary oxalate excretion. In certain embodiments, normal oxalate urinary excretion is less than about 0.45 mmol/1.73 m² per day, less than about 0.46 mmol/1.73 m² per day or less than about 0.5 mmol/1.73 m² per day. In certain embodiments, elevated oxalate levels means having a urinary oxalate excretion rate that is greater than about 40 mg/day. In certain embodiments, elevated oxalate levels means having a urinary oxalate excretion rate that is greater than about 45 mg/day. In certain embodiments, the urinary oxalate excretion rate is about two-fold higher than normal. In certain embodiments, the urinary oxalate excretion rate is about four-fold higher than normal. In certain embodiments, elevated oxalate levels means having a plasma oxalate levels greater than normal plasma oxalate levels of about 1 μmol/L to about 3 μmol/L. In certain embodiments, elevated oxalate levels means having a plasma oxalate level equal to or greater than about 10 μmol/L. In certain embodiments, elevated oxalate levels means having a plasma oxalate level equal to or greater than about 20 μmol/L.

Method of treating primary hyperoxaluria may include the step of selecting patients with the genetic mutation underlying PH1, PH2 or PH3, for example, using a diagnostic test to detect the presence of mutation in the AGXT, GRHPR, HOGA1 genes, or to detect the level of expression or activity of the AGXT, GRHPR, HOGA1 genes, before administering any of the compound or composition provided herein. Hyperoxaluria patients may also be diagnosed by kidney stone biopsy, measurement of urinary levels of oxalate, calcium, citrate, sodium, magnesium, urate, urinary pH and volume, or a combination of any such measurements, prior to administering a compound or composition provided herein.

Efficacy of the agent can be measured in a patient by reduction in the plasma or urinary oxalate, for example, in the course of days, weeks, months or years. Both plasma and urinary oxalate can be measured in patients in several ways, including concentration or mg of oxalate, moles of oxalate or concentration of oxalate in the biological media (urine or plasma). In addition, oxalate can be normalized to other proteins, such as creatinine, or evaluated over a 24 hour period, or normalized based on age, body mass or body surface area.

In certain embodiments, provided herein are methods of treating a disease or disorder associated with elevated oxalate levels, comprising administering to a subject having such disease or disorder, a therapeutically effective amount of a compound disclosed herein, or a pharmaceutically acceptable salt or solvate thereof, or a pharmaceutical composition thereof. In certain embodiments, the elevated oxalate levels is elevated urinary oxalate levels. In certain embodiments, the elevated oxalate levels is elevated plasma oxalate levels. In certain embodiments, the disease or disorder is hyperoxaluria, chronic kidney disease (CKD), end stage renal disease (ESRD) or kidney stone disease. In certain embodiments, the disease or disorder associated with elevated oxalate levels is hyperoxaluria. In certain embodiments, the hyperoxaluria is primary hyperoxaluria or secondary hyperoxaluria. In certain embodiments, the disease or disorder associated with elevated oxalate levels is primary hyperoxaluria, idiopathic hyperoxaluria or idiopathic oxalate kidney stone disease. In yet certain embodiments, the primary hyperoxaluria is primary hyperoxaluria type 1 (PH-1), primary hyperoxaluria type 2 (PH-2) or primary hyperoxaluria type 3 (PH-3). In yet certain embodiments, the disease or disorder associated with elevated oxalate levels is associated with AGXT, GRHPR or HOGA1 mutation, or a combination of mutations thereof.

In certain embodiments, provided herein are methods of treating hyperoxaluria, comprising administering to a subject having such disease or disorder, a therapeutically effective amount of a compound provided herein, or a pharmaceutically acceptable salt or solvate thereof, or a pharmaceutical composition thereof. In certain embodiments, provided herein are methods of treating hyperoxaluria, chronic kidney disease (CKD), end stage renal disease (ESRD) or kidney stone disease, comprising administering to a subject having such disease or disorder, a therapeutically effective amount of a compound disclosed herein, or a pharmaceutically acceptable salt or solvate thereof, or a pharmaceutical composition thereof.

In certain embodiments, provided herein are methods of treating primary hyperoxaluria type 1 (PH-1), primary hyperoxaluria type 2 (PH-2) or primary hyperoxaluria type 3 (PH-3), comprising administering to a subject having such disease or disorder, a therapeutically effective amount of a compound disclosed herein, or a pharmaceutically acceptable salt or solvate thereof, or a pharmaceutical composition thereof. In certain embodiments, provided herein are methods of treating disease or disorder associated with an AGXT, GRHPR or HOGA1 mutation, or a combination of mutations thereof, comprising administering to a subject having such disease or disorder, a therapeutically effective amount of a compound disclosed herein, or a pharmaceutically acceptable salt or solvate thereof, or a pharmaceutical composition thereof. In certain embodiments, provided herein are methods of lowering oxalate levels in a subject in need thereof, comprising administering to the subject a therapeutically effective amount of a compound disclosed herein, or a pharmaceutically acceptable salt or solvate thereof, or a pharmaceutical composition thereof. In certain embodiments, provided herein are methods of treating kidney stone formation in a subject in need thereof comprising administering to the subject a therapeutically effective amount of a compound disclosed herein, or a pharmaceutically acceptable salt or solvate thereof, or a pharmaceutical composition thereof.

In certain embodiments, provided herein are compounds disclosed herein, or a pharmaceutically acceptable salt or solvate thereof, or a pharmaceutical composition thereof, for use in treating a disease or disorder associated with elevated oxalate levels. In certain embodiments, provided herein are compounds disclosed herein, or a pharmaceutically acceptable salt or solvate thereof, or a pharmaceutical composition thereof, for use in treating hyperoxaluria, chronic kidney disease (CKD), end stage renal disease (ESRD) or kidney stone disease.

In certain embodiments, provided herein are compounds disclosed herein, or a pharmaceutically acceptable salt or solvate thereof, or a pharmaceutical composition thereof, for use in treating hyperoxaluria. In certain embodiments, provided herein are compounds disclosed herein, or a pharmaceutically acceptable salt or solvate thereof, or a pharmaceutical composition thereof, for use in treating primary hyperoxaluria or secondary hyperoxaluria.

In certain embodiments, the compounds disclosed herein, or a pharmaceutically acceptable salt or solvate thereof, or a pharmaceutical composition thereof, may be used in a variety of combination therapies to treat the conditions, diseases and disorders described above. Thus, also contemplated herein is the use of the compound disclosed herein, or a pharmaceutically acceptable salt or solvate thereof, or a pharmaceutical composition thereof, in combination with an additional pharmaceutical agent for the treatment of the conditions, diseases and disorders described herein. Additional agents which may be utilized for co-administration with the compound or composition provided herein, for example, an additional agent that lowers glyoxylate or oxalate levels, such as an RNAi therapeutic targeting GO expression (e.g. lumasiran (ALN-GO1), Alnylam's GalNAc-siRNA conjugates targeting GO) RNAi therapeutic targeting LDHA (e.g. nedosiran, Dicerna's GalNAc-siRNA conjugates targeting LDHA), other inhibitors in the oxalate synthesis pathways (e.g. stiripentol a weak LDH-inhibitor), or agents capable of reducing exogenous oxalate, such as oxalate decarboxylase (e.g. reloxaliase, formerly ALLN-177) or the oxalate degrading bacteria, oxalobacter formigenes (e.g. Oxabact®). The compound or composition provided herein may also be administered in conjunction with dietary modifications such as increased water consumption or avoidance of oxalate-rich food. The compound of composition provided herein may also be co-administered with vitamin B6 (pyridoxine), phosphate, magnesium or citrate.

In certain embodiments, provided herein are methods of treating the diseases or disorders described herein, further comprising administering to the subject in need thereof a therapeutically effective amount of a second therapeutic agent. In certain embodiments, the second therapeutic agent is a glyoxylate or oxalate lowering therapeutic. In certain embodiments, the glyoxylate or oxalate lowering therapeutic is an RNAi therapeutic. In yet certain embodiments, the glyoxylate or oxalate lowering therapeutic is lumasiran, nedosiran, reloxaliase, stiripentol, oxalobacter formigenes or vitamin B6.

### F. Preparation of the Compounds

The starting materials used for the synthesis were synthesized according to known literature procedures or obtained from commercial sources, such as, but not limited to, Sigma-Aldrich, Fluka, Acros Organics, Alfa Aesar, VWR Scientific, and the like. Nuclear Magnetic Resonance (NMR) analysis was conducted using a Bruker Acuity 300 MHz or 400 MHZ spectrometer with an appropriate deuterated solvent. Chemical shift (8) is expressed in units of parts per million (ppm). LCMS analysis was conducted using a Waters Acquity UPLC with a QDA MS detector using a Waters C18 BEH 1.7 μM, 2.1×50 mm column, eluting with 95:5 to 0:100 H₂O:MeCN+0.1% formic acid at a flow rate of 0.6 mL/min over 3.5 minutes, or using a Shimadzu LCMS-2020 using a Ascentis Express C18 2.7 μM, 3.0×50 mm column, eluting with 95:5 to 0:100 H₂O:MeCN+0.05% trifluoroacetic acid at a flow rate of 1.5 mL/min over 3.0 minutes. The QDA MS detector was set up to scan under both positive and negative mode ions ranging from 100-1200 Daltons. General methods for the preparation of compounds can be modified by the use of appropriate reagents and conditions for the introduction of the various moieties found in the structures as provided herein.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. Various alternatives to the embodiments of the disclosure described herein may be employed in practicing the disclosure. It is intended that the following claims define the scope of the disclosure and that methods and structures within the scope of these claims and their equivalents be covered thereby.

Standard abbreviations and acronyms as defined in Journal of Organic Chemistry's Author's Guideline at https://pubs.acs.org/userimages/ContentEditor/1218717864819/joceah_abbreviations.pdf are used herein. Other abbreviations and acronyms used herein are as follows:

TABLE 1
Abbreviations

| Ac | acetate |
|---|---|
| AIBN | 2,2′-azo-bis(2-methylpropionitrile) |
| aq. | aqueous |
| C | Celsius |
| Dabco ® 33-LV | 1,4-diazabicyclo[2.2.2]octane |
| | solution, 33 wt. % |
| | solution of triethylenediamine |
| | in dipropylene glycol |
| DABCO | 1,4-diazabicyclo[2.2.2]octane |
| DMF | dimethylformamide |
| DMSO | dimethylsulfoxide |
| EtOAc | ethyl acetate |
| Et | ethyl |
| equiv | equivalents |
| h | hours |
| Hunig′s base | N,N-diisopropylethylamine |
| g | grams |
| L | liter |
| LCMS | liquid chromatography-mass spectrometry |
| liq. | liquid |
| M | molar |
| Me | methyl |
| MeCN | acetonitrile |
| m-CPBA | meta-chloroperbenzoic acid |
| mg | milligram(s) |
| mL | milliliter(s) |
| mm | millimeter(s) |
| mmol | millimole(s) |
| mol | moles |
| MS | mass spectrometry |
| NBS | N-bromosuccinimide |
| nm | nanometer(s) |
| [PdCl(ally1)]₂ | allylpalladium(II) chloride dimer |
| PMB | p-methoxybenzyl |
| Pr | propyl |
| sat. | saturated |
| SEM | 2-(trimethylsilyl)ethoxymethyl |
| THF | tetrahydrofuran |
| TFA | trifluoroacetic acid |
| μL | microliter |
| μW | microwave reactor |
| μm | micrometer(s) |
| wt. | weight |

### General Synthetic Scheme

In some embodiments, compounds described herein can be prepared as outlined in the following general synthetic schemes.

### Method A: Sonogashira Coupling

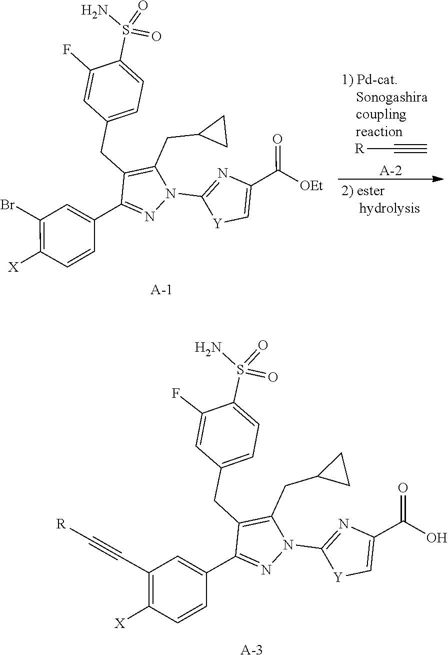

The bromo-intermediate A-1 (where X may be hydrogen or fluoro) can react with various readily available terminal alkynes A-2 (wherein R may be optionally substituted alkyl, optionally substituted cycloalkyl or optionally substituted heterocyclylalkyl) via a palladium-catalyzed Sonagashira coupling reaction, resulting in the alkynyl product (structure not shown). The corresponding carboxylate can undergo hydrolysis under ester saponification conditions, giving desired target compounds A-3.

### Method B: S_{N}Ar Reaction

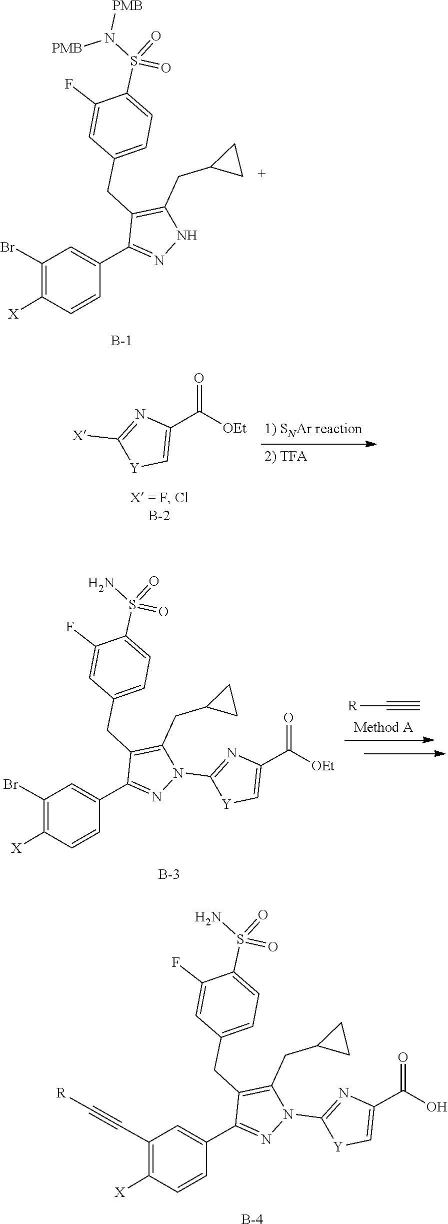

Pyrazole intermediate B-1 can react with a F- or Cl-heteroaryl carboxylate B-2, via an S_{N}Ar reaction. The intermediate generated (structure not shown) is treated with a strong acid such as TFA to remove the PMB protecting group and yielding the corresponding free sulfonamide B-3. Following the same procedure as Method A, intermediate B-3 can be converted to the final alkynyl product B-4.

### G. Examples

### Preparation of Intermediates

### Intermediate A: Ethyl 2-(3-(3-bromo-4-fluorophenyl)-5-(cyclopropylmethyl)-4-(3-fluoro-4-sulfamoylbenzyl)-1H-pyrazol-1-yl)thiazole-4-carboxylate

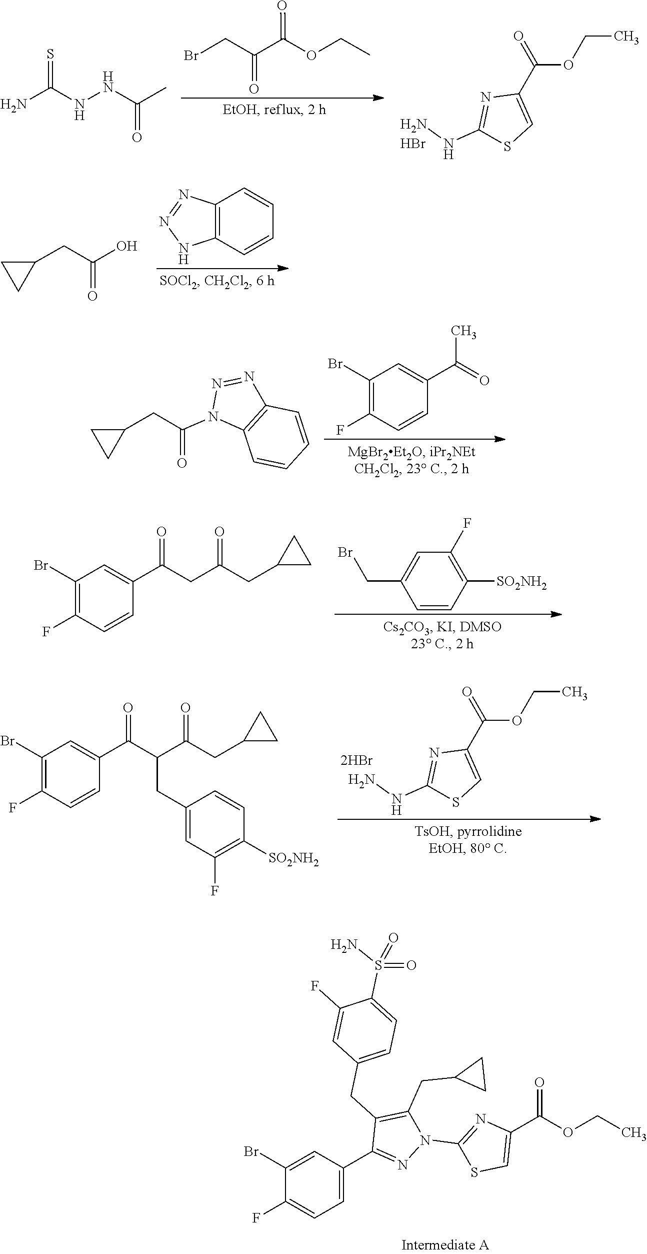

### Step 1: Preparation of ethyl 2-hydrazinylthiazole-4-carboxylate hydrobromide

To a stirred solution of N-(carbamothioylamino)acetamide (1.0 equiv) in EtOH (0.3 M) at 23° C. was added ethyl 3-bromo-2-oxopropanoate (1.0 equiv). The mixture was stirred at this temperature for 30 mins then heated to reflux for 2 h. The resulting mixture was concentrated under vacuum. The residue was purified by trituration with MeOH/Et₂O (1/6, v/v) to afford the title product as a yellow solid (49% yield).

### Step 2: Preparation of 1-(1H-benzo[d][1,2,3]triazol-1-yl)-2-cyclopropylethan-1-one

To a stirred solution of benzotriazole (4.0 equiv) in CH₂Cl₂ (0.3 M) at 23° C. was added thionyl chloride dropwise (1.0 equiv). The resulting mixture was stirred at this temperature for 30 mins. To this mixture was added cyclopropylacetic acid dropwise (1.0 equiv). The mixture was stirred for another 6 h. The resulting suspension was filtered. The filtrate was washed with aqueous sat. NaHCO₃ and brine. The organic layer was dried over Na₂SO₄, filtered and concentrated under vacuum to afford the title product as a red oil (63% yield, 72% purity by LCMS), which was used directly in the next step without further purification.

### Step 3: Preparation of 1-(3-bromo-4-fluorophenyl)-4-cyclopropylbutane-1,3-dione

To a stirred mixture of 1-(1,2,3-benzotriazol-1-yl)-2-cyclopropylethanone (1.0 equiv) in CH₂Cl₂ (0.3 M) at 23° C. was added magnesium bromide ethyl etherate (2.5 equiv) and 1-(3-bromo-4-fluorophenyl) ethanone (1.0 equiv). The resulting mixture was stirred at this temperature for 10 mins. To this mixture was added N,N-diisopropylethylamine dropwise (3.0 equiv). The resulting mixture was stirred for another 2 h. The mixture was acidified with 1M aqueous HCl solution until the pH was ˜1 and the mixture was extracted with CH₂Cl₂ (3×3 volumes). The organic layers were combined, washed with saturated aqueous NaHCO₃ solution and brine, dried over Na₂SO₄, and concentrated under vacuum. The residue was purified by column chromatography through silica gel, eluting with 0% to 5% EtOAc in petroleum ether as a gradient, to afford the title product as a yellow solid (51% yield).

### Step 4: Preparation of 4-(2-(3-bromo-4-fluorobenzoyl)-4-cyclopropyl-3-oxobutyl)-2-fluorobenzenesulfonamide

A mixture of 1-(3-bromo-4-fluorophenyl)-4-cyclopropylbutane-1,3-dione (1.0 equiv), 4-(bromomethyl)-2-fluorobenzenesulfonamide (1.1 equiv), Cs₂CO₃ (1.5 equiv) and potassium iodide (1.0 equiv) in DMSO (1.1 M) was stirred at 23° C. for 2 h then diluted with EtOAc. The organic layer was washed with 1M aqueous HCl solution, brine, dried over Na₂SO₄, filtered and concentrated under vacuum. The residue was purified by column chromatography through silica gel, eluting with 0% to 30% EtOAc in petroleum ether, to afford the title product as a yellow solid (64% yield).

### Step 5: Preparation of ethyl 2-(3-(3-bromo-4-fluorophenyl)-5-(cyclopropylmethyl)-4-(3-fluoro-4-sulfamoylbenzyl)-1H-pyrazol-1-yl)thiazole-4-carboxylate

To a stirred solution of 4-[2-(3-bromo-4-fluorobenzoyl)-4-cyclopropyl-3-oxobutyl]-2-fluorobenzenesulfonamide (1.0 equiv) in EtOH (0.2 M) at 23° C. was added pyrrolidine (0.3 equiv) and p-toluenesulfonic acid (0.5 equiv). The resulting mixture was stirred at this temperature for 30 mins. To this mixture was added ethyl 2-hydrazinyl-1,3-thiazole-4-carboxylate dihydrobromide (1.2 equiv). The reaction was stirred at 80° C. under nitrogen for 2 h. Upon completion of reaction, the mixture was concentrated under vacuum. The residue was purified by reverse phase column chromatography on C18, eluting with 95:5 to 15:85 H₂O:MeCN+0.1% formic acid as a gradient to afford the title product as a light yellow solid (15% yield).

### Intermediate B: Ethyl 2-(3-(3-bromophenyl)-5-(cyclopropylmethyl)-4-(3-fluoro-4-sulfamoylbenzyl)-1H-pyrazol-1-yl)thiazole-4-carboxylate

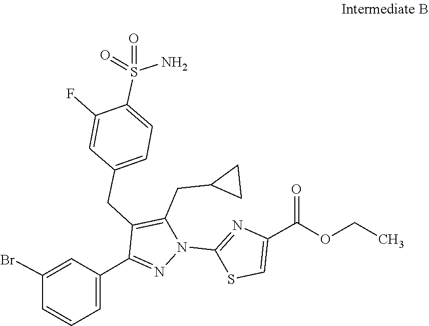 [SMILES: CCOC(=O)c1csc(-n2nc(-c3cccc(Br)c3)c(Cc3ccc(S(N)(=O)=O)c(F)c3)c2CC2CC2)n1]

Intermediate B was prepared in the same manner as Intermediate A utilizing 3-bromoacetophenone as a starting material in step 3.

### Intermediate C: 4-(Bromomethyl)-2-fluoro-N,N-bis(4-methoxybenzyl)benzenesulfonamide

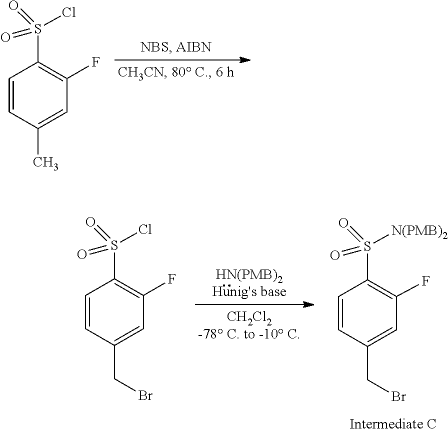

### Step 1: Preparation of 4-(bromomethyl)-2-fluorobenzenesulfonyl chloride

To a degassed solution of 2-fluoro-4-methyl-benzenesulfonyl chloride (1.0 equiv) in MeCN (0.4 M) was added N-bromosuccinimide (1.1 equiv), 2,2′-azo-bis(2-methylpropionitrile) (0.1 equiv) and the mixture was heated to 80° C. in an oil bath for 8 hours. This mixture was concentrated under reduced vacuum. The resulting oil was purified by column chromatography through silica gel, eluting with 0 to 10% EtOAc in hexanes as a gradient. Select fractions were monitored by TLC (EtOAc: hexanes 1:9, UV). The desired fractions from the major peak which elutes centered at 7% EtOAc in hexanes were combined and concentrated to provide the title compound as a white crystalline solid (53% yield).

### Step 2: Preparation of 4-(bromomethyl)-2-fluoro-N,N-bis(4-methoxybenzyl)benzenesulfonamide

To a solution of 4-(bromomethyl-2-fluorobenzenesulfonyl chloride (1.0 equiv) in CH₂Cl₂ (0.3 M) under nitrogen and cooled in a dry ice/acetone bath was added N-(4-methoxybenzyl)-1-(4-methoxyphenyl)methanamine (1.0 equiv). Once cooled, diisopropylethylamine (1.1 equiv) was added portion-wise over 10 minutes and the mixture was allowed to stir in the dry ice/acetone bath for 30 minutes. After this time, the cooling bath was exchanged with a salt/wet ice bath (−10° C.) and stirring was continued for 2 hours. This mixture was quenched with ice (1 volume), water (1 volume) and then extracted with CH₂Cl₂ (2×1 volume). The combined organic extracts were washed with water (0.25 volumes), dried over MgSO₄, filtered and concentrated under vacuum to afford an oil. The resulting oil was dissolved in CH₂Cl₂ (0.1 volumes), applied to a silica gel pre-cartridge and purified by column chromatography through silica gel, eluting with 0-20% EtOAc in CH₂Cl₂ as a gradient to provide the title compound as a white solid (73% yield).

### Intermediate D: 4-((3-(3-Bromo-4-fluorophenyl)-5-(cyclopropylmethyl)-1H-pyrazol-4-yl)methyl)-2-fluoro-N,N-bis(4-methoxybenzyl)benzenesulfonamide

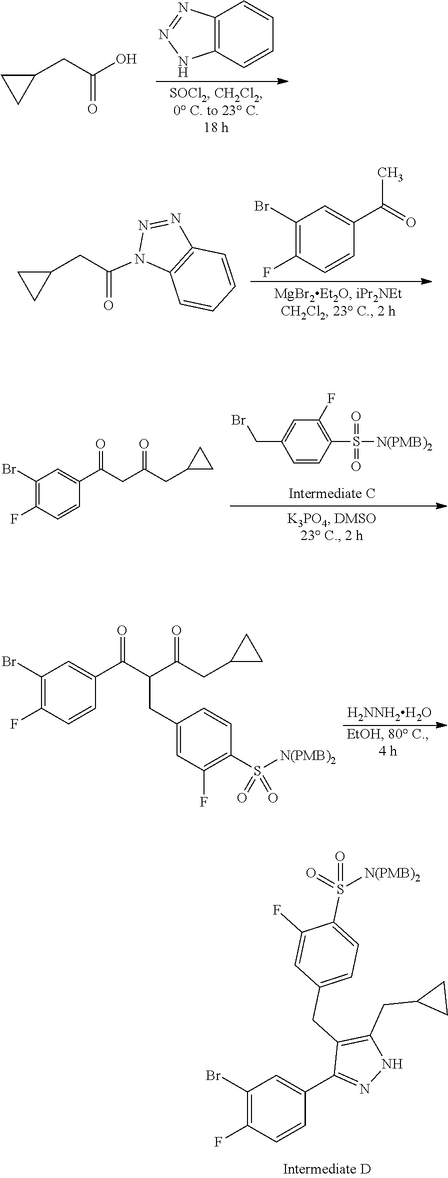

### Step 1: Preparation of 1-(1H-benzo[d][1,2,3]triazol-1-yl)-2-cyclopropylethan-1-one

Into a round-bottom flask equipped with a magnetic stir bar and under N₂ was added benzotriazole (4.0 equiv) and CH₂Cl₂ (0.3 M). The resulting clear solution was cooled to 0° C. in an ice bath and thionyl chloride (1.0 equiv) was added drop-wise over 10 minutes to the cooled solution. The resulting light-yellow solution was warmed and stirred at 23° C. for 1 h and then cooled back down to 0° C. in an ice bath. Cyclopropylacetic acid (1.0 equiv) was added in a single portion and the resulting white suspension was stirred at 23° C. for 16 h overnight. The resulting white suspension was filtered through Whatman #1 filter paper on a Hirsch funnel under vacuum, washing with CH₂Cl₂ (3×). The clear filtrate was concentrated under reduced pressure and loaded onto a silica gel pre-cartridge and dried under vacuum. The mixture was purified by column chromatography through silica gel, eluting with 95:5 to 50:50 hexanes: EtOAc as a gradient over 25 minutes, collecting all peaks. The desired product containing fractions were concentrated and dried under vacuum to afford a clear oil which crystallized slowly upon standing (81% yield).

### Step 2: Preparation of 1-(3-bromo-4-fluorophenyl)-4-cyclopropylbutane-1,3-dione

Into a round-bottom flask equipped with a magnetic stir bar was added 1-(benzotriazol-1-yl)-2-cyclopropyl-ethanone (1.2 equiv), 1-(3-bromo-4-fluorophenyl)ethan-1-one (1.0 equiv) and CH₂Cl₂ (0.3 M). The mixture was cooled to 0° C. in an ice bath. With stirring, magnesium bromide ethyl etherate (2.5 equiv) was in a single portion followed by N,N-diisopropylethylamine (3.0 equiv) drop-wise via an additional funnel. Upon addition, the reaction mixture turned yellow, as the solid started to dissolve. The ice bath was removed when the addition was complete and the reaction was allowed to warm to 23° C. and stirred for another 30 min. LC-MS analysis indicated completion of reaction. The reaction was cooled back to 0° C. in ice bath, and slowly quenched with the addition of 1 M aqueous HCl solution to pH˜2. The resulting aqueous mixture was partitioned in a separatory funnel. The aqueous layer was extracted with CH₂Cl₂ (3×). The combined organic layers were washed with brine, dried with MgSO₄, filtered and concentrated under reduced pressure. The resulting crude reaction mixture was loaded onto a silica gel pre-cartridge and purified by column chromatography through silica gel, eluting with 0:100 to 80:20 hexanes: EtOAc as a gradient over 30 min. The desired product was eluted from 90:10 to 80:20 hexanes: EtOAc. The desired product containing fractions were concentrated and dried under vacuum to afford a yellow oil (64% yield).

### Step 3: Preparation of 4-(2-(3-bromo-4-fluorobenzoyl)-4-cyclopropyl-3-oxobutyl)-2-fluoro-N,N-bis(4-methoxybenzyl)benzenesulfonamide

Into a round-bottom flask equipped with a magnetic stir bar was added 1-(3-bromo-4-fluorophenyl)-4-cyclopropylbutane-1,3-dione (1.0 equiv) and DMSO (0.3 M). The solution was treated with potassium phosphate (1.5 equiv) and the resulting suspension was stirred for 30 minutes at room temperature. At this time, the mixture was cooled to 0° C. in an ice bath and 4-(bromomethyl)-2-fluoro-N,N-bis(4-methoxybenzyl)benzenesulfonamide (Intermediate C, 1.2 equiv) was added. The mixture was stirred at 23° C. for 2 h and upon completion of reaction, the reaction mixture was poured into a separatory funnel and extracted with ethyl acetate (2×). The combined organic layers were washed with brine (2×), dried over anhydrous Na₂SO₄, and filtered. The filtrate was concentrated under reduced pressure and purified by column chromatography through silica gel eluting with 95:5 to 70:30 petroleum ether:ethyl acetate as a gradient over 20 min, collecting all peaks. The desired product containing fractions were concentrated to afford the title product as a reddish brown yellow oil (49% yield).

### Step 4: Preparation of 4-((3-(3-bromo-4-fluorophenyl)-5-(cyclopropylmethyl)-1H-pyrazol-4-yl)methyl)-2-fluoro-N,N-bis(4-methoxybenzyl)benzenesulfonamide

Into a round bottom flask equipped with a magnetic stir bar was added 4-(2-(3-bromo-4-fluorobenzoyl)-4-cyclopropyl-3-oxobutyl)-2-fluoro-N,N-bis(4-methoxybenzyl)benzenesulfonamide (1.0 equiv) and EtOH (0.14 M). Concentrated hydrazine hydrate (5.0 equiv) was added to the flask and the solution was heated to 80° C. in an oil bath for 4 h. LC-MS analysis indicated consumption of starting material and formation of product. The reaction mixture was cooled to room temperature and concentrated under reduced pressure. The resulting yellow oil was loaded into a pre-cartridge and dried. The mixture was purified by column chromatography through silica gel eluting with 0:100 to 60:40 petroleum ether:ethyl acetate as a gradient over 20 min to afford the title product as a yellow solid (61% yield).

### PREPARATION OF EXAMPLES

### Example 1: Preparation of 2-(5-(Cyclopropylmethyl)-3-(4-fluoro-3-((3-hydroxyoxetan-3-yl)ethynyl)phenyl)-4-(3-fluoro-4-sulfamoylbenzyl)-1H-pyrazol-1-yl)thiazole-4-carboxylic acid

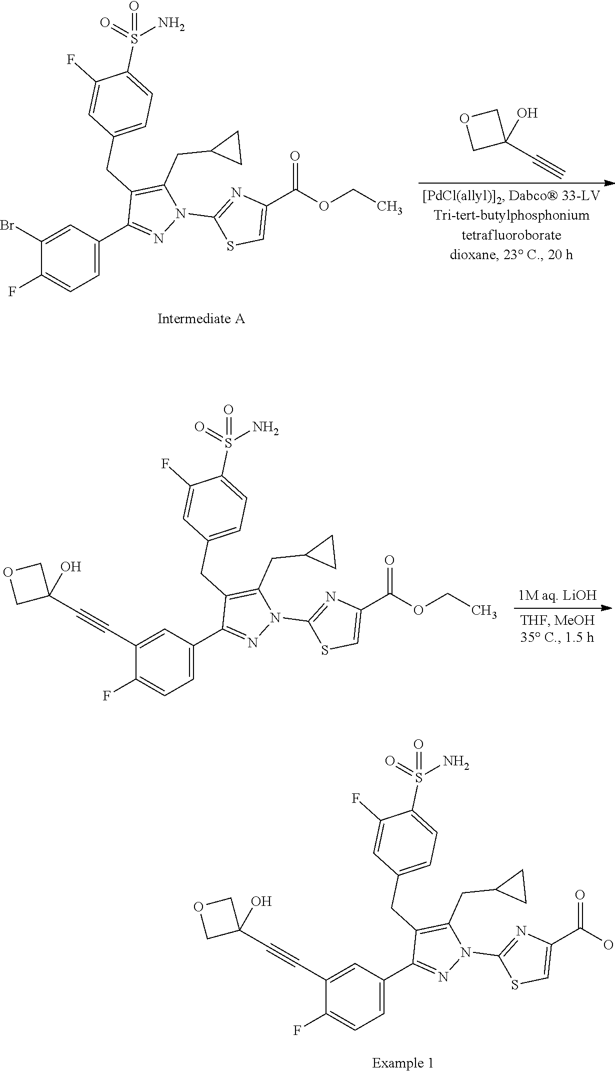

### Step 1: Preparation of ethyl 2-(5-(cyclopropylmethyl)-3-(4-fluoro-3-((3-hydroxyoxetan-3-yl)ethynyl)phenyl)-4-(3-fluoro-4-sulfamoylbenzyl)-1H-pyrazol-1-yl)thiazole-4-carboxylate

Into a round bottom flask equipped with a magnetic stir bar was added ethyl 2-(3-(3-bromo-4-fluorophenyl)-5-(cyclopropylmethyl)-4-(3-fluoro-4-sulfamoylbenzyl)-1H-pyrazol-1-yl)thiazole-4-carboxylate (Intermediate A, 1.0 equiv), 3-ethynyloxetan-3-ol (10 equiv), Dabco® 33-LV (3.0 equiv), tri-tert-butylphosphonium tetrafluoroborate (0.3 equiv), allylpalladium (II) chloride dimer (0.15 equiv) and dioxane (0.05 M). The reaction mixture was degassed for 10 minutes and stirred at 23° C. for 20 hours. Purification by reverse-phase column chromatography using a C18 column, eluting with 90:10 to 0:100 H₂O:MeCN+0.1% formic acid as a gradient. Fractions containing the desired product were concentrated under reduced pressure to afford the title compound (75% yield).

### Step 2: Preparation of 2-(5-(cyclopropylmethyl)-3-(4-fluoro-3-((3-hydroxyoxetan-3-yl)ethynyl)phenyl)-4-(3-fluoro-4-sulfamoylbenzyl)-1H-pyrazol-1-yl)thiazole-4-carboxylic acid

Into a round bottom flask equipped with a magnetic stir bar was added ethyl 2-(5-(cyclopropylmethyl)-3-(4-fluoro-3-((3-hydroxyoxetan-3-yl)ethynyl)phenyl)-4-(3-fluoro-4-sulfamoylbenzyl)-1H-pyrazol-1-yl)thiazole-4-carboxylate (1.0 equiv), THF (0.18 M) and MeOH (0.18 M). A solution of 1M aqueous LiOH (8.5 equiv) was added and the mixture was stirred at 35° C. for 90 minutes. Purification by reverse-phase column chromatography using a C18 column, eluting with 90:10 to 0:100 H₂O:MeCN+0.1% formic acid as a gradient. Fractions containing the desired product were concentrated under reduced pressure to afford the title compound (91% yield). ¹H NMR (400 MHZ, do-DMSO) δ_{H} 8.24 (s, 1H), 7.70 (d, J=7.0 Hz, 1H), 7.67-7.55 (m, 4H), 7.34 (t, J=9.0 Hz, 1H), 7.13 (d, J=11.0 Hz, 1H), 7.03 (d, J=8.0 Hz, 1H), 6.71 (s, 1H), 4.74 (d, J=6.5 Hz, 2H), 4.59 (d, J=6.5 Hz, 2H), 4.15 (s, 2H), 3.15 (d, J=7.0 Hz, 2H), 1.16-1.05 (m, 1H), 0.34-0.28 (m, 2H), 0.22-0.16 (m, 2H).

| Example | Structure | MW | MS (ESI+) |
|---|---|---|---|
| Example 1 | 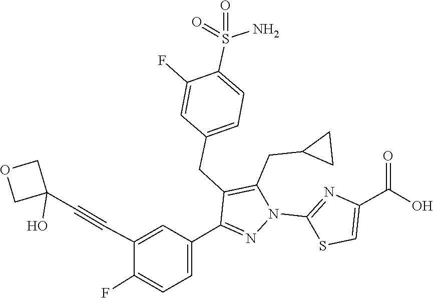 [SMILES: NS(=O)(=O)c1ccc(Cc2c(-c3ccc(F)c(C#CC4(O)COC4)c3)nn(-c3nc(C(=O)O)cs3)c2CC2CC2)cc1F] | 626.11 | 627 (M + 1)^{⊕} |
| | 2-(5-(cyclopropylmethyl)-3-(4-fluoro-3-((3- | | |
| | hydroxyoxetan-3-yl)ethynyl)phenyl)-4-(3-fluoro-4- | | |
| | sulfamoylbenzyl)-1H-pyrazol-1-yl)thiazole-4-carboxylic | | |
| | acid | | |

### Example 2: Preparation of 2-(5-(Cyclopropylmethyl)-3-(4-fluoro-3-(3-hydroxy-3-methylbut-1-yn-1-yl)phenyl)-4-(3-fluoro-4-sulfamoylbenzyl)-1H-pyrazol-1-yl)thiazole-4-carboxylic acid

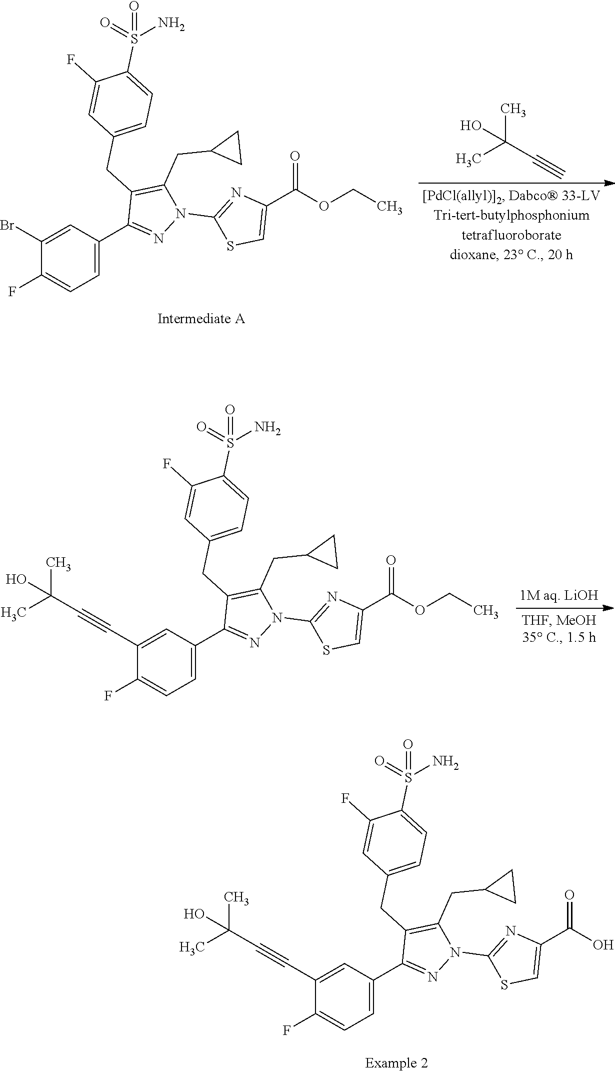

### Step 1: Preparation of ethyl 2-(5-(cyclopropylmethyl)-3-(4-fluoro-3-(3-hydroxy-3-methylbut-1-yn-1-yl)phenyl)-4-(3-fluoro-4-sulfamoylbenzyl)-1H-pyrazol-1-yl)thiazole-4-carboxylate

Into a round bottom flask equipped with a magnetic stir bar was added ethyl 2-(3-(3-bromo-4-fluorophenyl)-5-(cyclopropylmethyl)-4-(3-fluoro-4-sulfamoylbenzyl)-1H-pyrazol-1-yl)thiazole-4-carboxylate (Intermediate A, 1.0 equiv), 2-methylbut-3-yn-2-ol (10 equiv), Dabco® 33-LV (3.0 equiv), tri-tert-butylphosphonium tetrafluoroborate (0.3 equiv), allylpalladium (II) chloride dimer (0.15 equiv) and dioxane (0.05 M). The reaction mixture was degassed for 10 minutes and stirred at 23° C. for 20 hours. Purification by reverse-phase column chromatography using a C18 column, eluting with 90:10 to 0:100 H₂O:MeCN+0.1% formic acid as a gradient. Fractions containing the desired product were concentrated under reduced pressure to afford the title compound (90% yield).

### Step 2: Preparation of 2-(5-(cyclopropylmethyl)-3-(4-fluoro-3-(3-hydroxy-3-methylbut-1-yn-1-yl)phenyl)-4-(3-fluoro-4-sulfamoylbenzyl)-1H-pyrazol-1-yl)thiazole-4-carboxylic acid

Into a round bottom flask equipped with a magnetic stir bar was added ethyl 2-(5-(cyclopropylmethyl)-3-(4-fluoro-3-((3-hydroxyoxetan-3-yl)ethynyl)phenyl)-4-(3-fluoro-4-sulfamoylbenzyl)-1H-pyrazol-1-yl)thiazole-4-carboxylate (1.0 equiv), THF (0.18 M) and MeOH (0.18 M). A solution of 1 M aqueous LiOH (8.5 equiv) was added and the mixture was stirred at 35° C. for 90 minutes. Purification by reverse-phase column chromatography using a C18 column, eluting with 90:10 to 0:100 H₂O:MeCN+0.1% formic acid as a gradient. Fractions containing the desired product were concentrated under reduced pressure to afford the title compound (98% yield). ¹H NMR (400 MHZ, do-DMSO) δ_{H} 13.20 (s, 1H), 8.28 (s, 1H), 7.66-7.52 (m, 5H), 7.30 (t, J=9.5 Hz, 1H), 7.14 (d, J=11.5 Hz, 1H), 7.03 (d, J=8.0 Hz, 1H), 5.54 (s, 1H), 4.13 (s, 2H), 3.14 (d, J=7.0 Hz, 2H), 1.44 (s, 6H), 1.16-1.07 (m, 1H), 0.35-0.29 (m, 2H), 0.24-0.17 (m, 2H).

| Example | Structure | MW | MS (ESI+) |
|---|---|---|---|
| Example 2 | 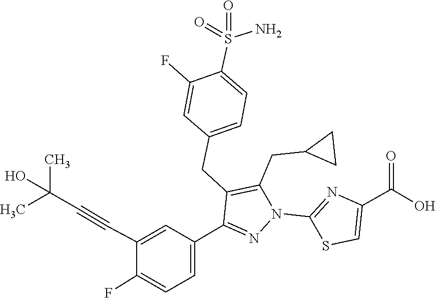 [SMILES: CC(C)(O)C#Cc1cc(-c2nn(-c3nc(C(=O)O)cs3)c(CC3CC3)c2Cc2ccc(S(N)(=O)=O)c(F)c2)ccc1F] | 612.13 | 613 (M + 1)^{⊕} |
| | 2-(5-(cyclopropylmethyl)-3-(4-fluoro-3-(3-hydroxy-3- | | |
| | methylbut-1-yn-1-yl)phenyl)-4-(3-fluoro-4-sulfamoylbenzyl)- | | |
| | 1H-pyrazol-1-yl)thiazole-4-carboxylic acid | | |

### Examples 3-9

The following examples, Example 3, Example 4 and Example 5, were prepared as per Example 2 by replacing 2-methylbut-3-yn-2-ol with corresponding alkynes in the first step. The following examples, Example 6, Example 7, Example 8 and Example 9, were prepared as in Example 2 by replacing Intermediate A and 2-methylbut-3-yn-2-ol with Intermediate B and the corresponding alkynes in the first step.

| Example | Structure | MW | MS (ESI+) |
|---|---|---|---|
| Example 3 | 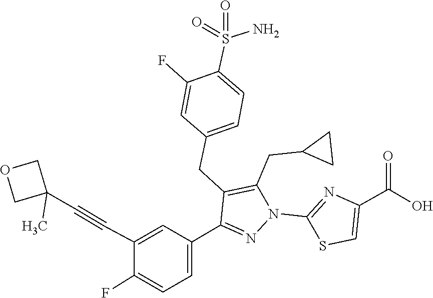 [SMILES: CC1(C#Cc2cc(-c3nn(-c4nc(C(=O)O)cs4)c(CC4CC4)c3Cc3ccc(S(N)(=O)=O)c(F)c3)ccc2F)COC1] | 624.13 | 625 (M + 1)^{⊕} |
| | 2-(5-(cyclopropylmethyl)-3-(4-fluoro-3-((3-methyloxetan-3- | | |
| | yl)ethynyl)phenyl)-4-(3-fluoro-4-sulfamoylbenzyl)-1H- | | |
| | pyrazol-1-yl)thiazole-4-carboxylic acid | | |
| Example 4 | 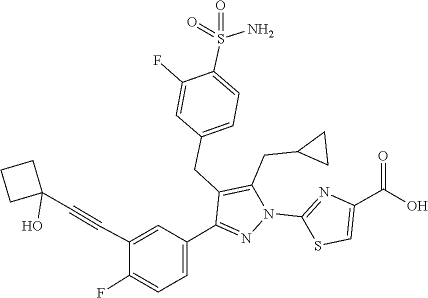 [SMILES: NS(=O)(=O)c1ccc(Cc2c(-c3ccc(F)c(C#CC4(O)CCC4)c3)nn(-c3nc(C(=O)O)cs3)c2CC2CC2)cc1F] | 624.13 | 625 (M + 1)^{⊕} |
| | 2-(5-(cyclopropylmethyl)-3-(4-fluoro-3-((1- | | |
| | hydroxycyclobutyl)ethynyl)phenyl)-4-(3-fluoro-4- | | |
| | sulfamoylbenzyl)-1H-pyrazol-1-yl)thiazole-4-carboxylic acid | | |
| Example 5 | 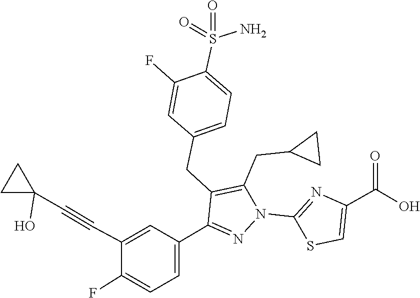 [SMILES: NS(=O)(=O)c1ccc(Cc2c(-c3ccc(F)c(C#CC4(O)CC4)c3)nn(-c3nc(C(=O)O)cs3)c2CC2CC2)cc1F] | 610.65 | 611 (M + 1)^{⊕} |
| | 2-(5-(cyclopropylmethyl)-3-(4-fluoro-3-((1- | | |
| | hydroxycyclopropl)ethynyl)phenyl)-4-(3-fluoro-4- | | |
| | sulfamoylbenzyl)-1H-pyrazol-1-yl)thiazole-4-carboxylic acid | | |
| Example 6 | 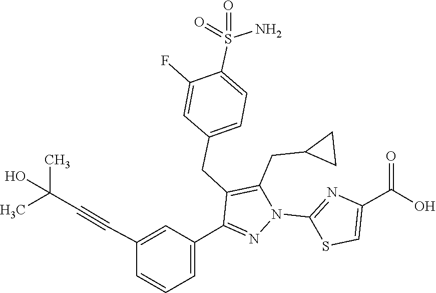 [SMILES: CC(C)(O)C#Cc1cccc(-c2nn(-c3nc(C(=O)O)cs3)c(CC3CC3)c2Cc2ccc(S(N)(=O)=O)c(F)c2)c1] | 594.14 | 595 (M + 1)^{⊕} |
| | 2-(5-(cyclopropylmethyl)-4-(3-fluoro-4-sulfamoylbenzyl)-3- | | |
| | (3-(3-hydroxy-3-methylbut-1-yn-1-yl)phenyl)-1H-pyrazol-1- | | |
| | yl)thiazole-4-carboxylic acid | | |
| Example 7 | 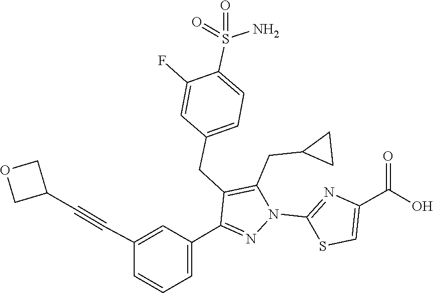 [SMILES: NS(=O)(=O)c1ccc(Cc2c(-c3cccc(C#CC4COC4)c3)nn(-c3nc(C(=O)O)cs3)c2CC2CC2)cc1F] | 592.13 | 593 (M + 1)^{⊕} |
| | 2-(5-(cyclopropylmethyl)-4-(3-fluoro-4-sulfamoylbenzyl)-3- | | |
| | (3-(oxetan-3-ylethynyl)phenyl)-1H-pyrazol-1-yl)thiazole-4- | | |
| | carboxylic acid | | |
| Example 8 | 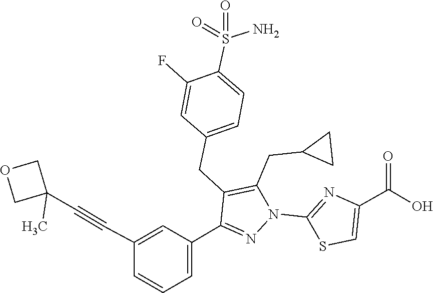 [SMILES: CC1(C#Cc2cccc(-c3nn(-c4nc(C(=O)O)cs4)c(CC4CC4)c3Cc3ccc(S(N)(=O)=O)c(F)c3)c2)COC1] | 606.14 | 607 (M + 1)^{⊕} |
| | 2-(5-(cyclopropylmethyl)-4-(3-fluoro-4-sulfamoylbenzyl)-3- | | |
| | (3-((3-methyloxetan-3-yl)ethynyl)phenyl)-1H-pyrazol-1- | | |
| | yl)thiazole-4-carboxylic acid | | |
| Example 9 | 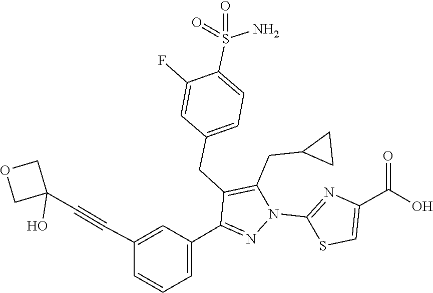 [SMILES: NS(=O)(=O)c1ccc(Cc2c(-c3cccc(C#CC4(O)COC4)c3)nn(-c3nc(C(=O)O)cs3)c2CC2CC2)cc1F] | 608.12 | 609 (M + 1)^{⊕} |
| | 2-(5-(cyclopropylmethyl)-4-(3-fluoro-4-sulfamoylbenzyl)-3- | | |
| | (3-((3-hydroxyoxetan-3-yl)ethynyl)phenyl)-1H-pyrazol-1- | | |
| | yl)thiazole-4-carboxylic acid | | |

### Example 10: Preparation of 2-(3-(3-(Cyclopropylethynyl)-4-fluorophenyl)-5-(cyclopropylmethyl)-4-(3-fluoro-4-sulfamoylbenzyl)-1H-pyrazol-1-yl)oxazole-4-carboxylic acid

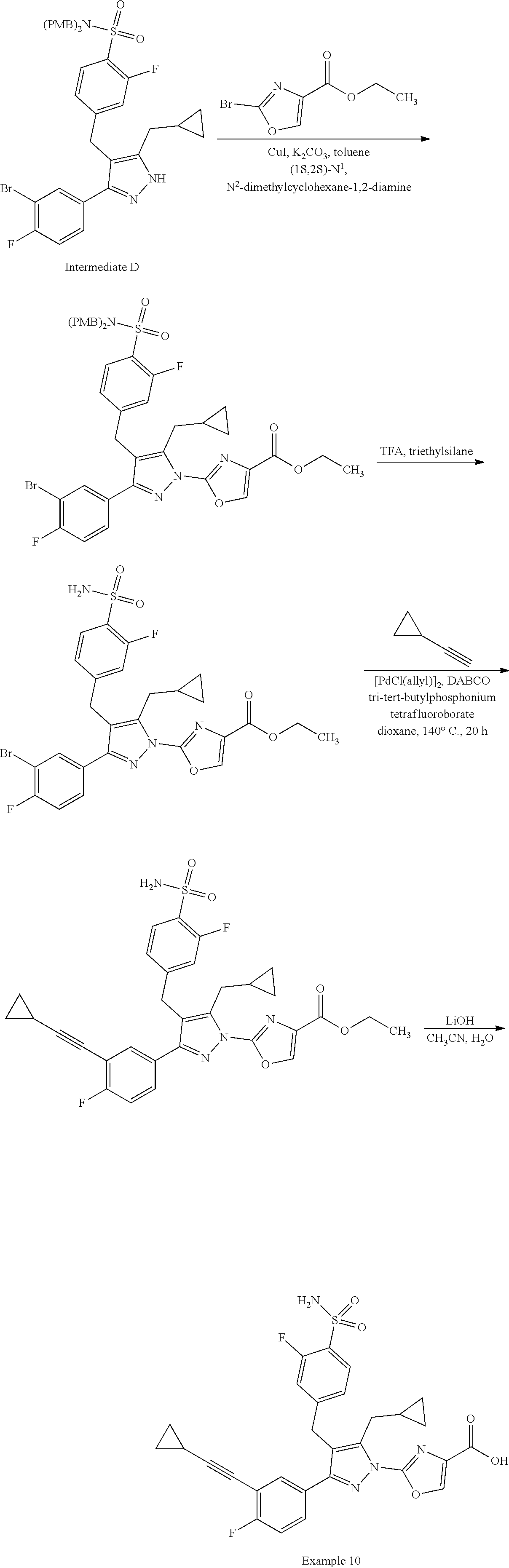

### Step 1: Preparation of ethyl 2-[4-[(4-[bis[(4-methoxyphenyl)methyl]sulfamoyl]-3-fluorophenyl)methyl]-3-(3-bromo-4-fluorophenyl)-5-(cyclopropylmethyl)pyrazol-1-yl]-1,3-oxazole-4-carboxylate

Into a round-bottom flask equipped with a magnetic stir bar was added 4-[[3-(3-bromo-4-fluorophenyl)-5-(cyclopropylmethyl)-1H-pyrazol-4-yl]methyl]-2-fluoro-N,N-bis[(4-methoxyphenyl)methyl]benzenesulfonamide (Intermediate D, 1.0 equiv), toluene (0.09 M), ethyl 2-bromo-1,3-oxazole-4-carboxylate (2.0 equiv), CuI (0.5 equiv), K₂CO₃ (3.0 equiv) and (1S,2S)-N¹,N²-dimethylcyclohexane-1,2-diamine (0.5 equiv). The resulting mixture was degassed with a steady flow of nitrogen for 10 minutes then heated to 120° C. in an oil bath under nitrogen atmosphere for 18 h overnight. LC-MS analysis indicated consumption of starting material and formation of product. The reaction mixture was cooled to 23° C. and quenched with water. The resulting mixture was poured into a separatory funnel and extracted with ethyl acetate (3×). The combined organic layers were washed with water, dried over anhydrous Na₂SO₄, filtered and concentrated under reduced pressure. The residue was purified by silica gel column chromatography, eluting with petroleum ether:ethyl acetate (3:2) to afford the title product as a yellow oil (50% yield).

### Step 2: Preparation of ethyl 2-[3-(3-bromo-4-fluorophenyl)-5-(cyclopropylmethyl)-4-[(3-fluoro-4-sulfamoylphenyl)methyl]pyrazol-1-yl]-1,3-oxazole-4-carboxylate

Into a round bottom flask equipped with a magnetic stir bar was added ethyl 2-[4-[(4-[bis[(4-methoxyphenyl)methyl]sulfamoyl]-3-fluorophenyl)methyl]-3-(3-bromo-4-fluorophenyl)-5-(cyclopropylmethyl)pyrazol-1-yl]-1,3-oxazole-4-carboxylate (1.0 equiv), TFA (0.5 M) and triethylsilane (0.125 M). The resulting mixture was stirred at 23° C. for 18 h overnight. LC-MS analysis indicated consumption of starting material and formation of product. The resulting mixture was concentrated under reduced pressure. The crude residue was purified by reverse flash chromatography through C18 eluting with 100:0 to 0:100 water:MeCN as a gradient over 35 minutes. The product containing fractions were combined, concentrated and dried under vacuum to afford the title product as a light yellow oil (37% yield).

### Step 3: Preparation of ethyl 2-[3-[3-(2-cyclopropylethynyl)-4-fluorophenyl]-5-(cyclopropylmethyl)-4-[(3-fluoro-4-sulfamoylphenyl)methyl]pyrazol-1-yl]-1,3-oxazole-4-carboxylate

Into a round bottom flask equipped with a magnetic stir bar was added ethyl 2-[3-(3-bromo-4-fluorophenyl)-5-(cyclopropylmethyl)-4-[(3-fluoro-4-sulfamoylphenyl)methyl]pyrazol-1-yl]-1,3-oxazole-4-carboxylate (1.0 equiv), DMSO (0.06 M), allylpalladium (II) chloride dimer (0.2 equiv), tri-tert-butyl-phosphonium tetrafluoroborate (0.2 equiv) and 1,4-diazabicyclo[2,2,2]octane (3.0 equiv). The resulting mixture was degassed with a steady flow of nitrogen for 10 minutes. To the above mixture was added ethynylcyclopropane (2.0 equiv) in DMSO (0.18 M) at room temperature. The resulting mixture heated to 140° C. in an oil bath and stirred for additional 2 h under a nitrogen atmosphere. LC-MS analysis indicated consumption of starting material and formation of product. The reaction mixture was cooled to 23° C. and quenched with water. The resulting mixture was extracted with ethyl acetate (3×). The combined organic layers were washed with brine and dried over anhydrous Na₂SO₄, filtered and concentrated under reduced pressure. The residue was purified by silica gel column chromatography, eluting with petroleum ether:ethyl acetate (4:1) to afford the title product as a white solid (16% yield).

### Step 4: Preparation of 2-[3-[3-(2-cyclopropylethynyl)-4-fluorophenyl]-5-(cyclopropylmethyl)-4-[(3-fluoro-4-sulfamoylphenyl)methyl]pyrazol-1-yl]-1,3-oxazole-4-carboxylic acid

Into a round bottom flask equipped with a magnetic stir bar was added ethyl 2-[3-[3-(2-cyclopropylethynyl)-4-fluorophenyl]-5-(cyclopropylmethyl)-4-[(3-fluoro-4-sulfamoylphenyl)methyl]pyrazol-1-yl]-1,3-oxazole-4-carboxylate (1.0 equiv), CH₃CN (0.03 M), H₂O (0.03 M) and solid LiOH (43 equiv). The resulting mixture was stirred at room temperature for 1 h. LC-MS analysis indicated consumption of starting material and formation of product. The resulting mixture was concentrated under reduced pressure. The residue was acidified to pH˜5 with 3 M aqueous HCl solution. The aqueous layer was extracted with ethyl acetate (3×) then dried over anhydrous Na₂SO₄. After filtration, the filtrate was concentrated under reduced pressure and the resulting mixture was concentrated. The crude product was purified by preparative HPLC with through a X-Select CSH OBD column, eluting with 50% to 66% MeCN in water over 8 minutes. The desired fractions were concentrated and further dried under vacuum to yield the title product as a white solid (27% yield).

| Example | Structure | MW | MS (ESI+) |
|---|---|---|---|
| Example 10 | 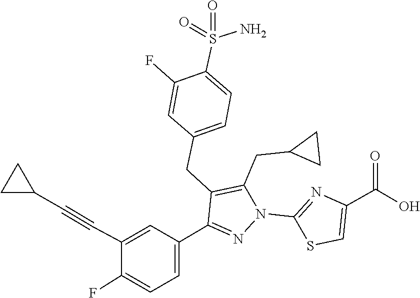 [SMILES: NS(=O)(=O)c1ccc(Cc2c(-c3ccc(F)c(C#CC4CC4)c3)nn(-c3nc(C(=O)O)cs3)c2CC2CC2)cc1F] | 578.59 | 579 (M + 1)^{⊕} |
| | 2-(3-(3-(cyclopropylethynyl)-4-fluorophenyl)-5- | | |
| | (cyclopropylmethyl)-4-(3-fluoro-4-sulfamoylbenzyl)-1H- | | |
| | pyrazol-1-yl)oxazole-4-carboxylic acid | | |

### Example 11: Human LDHA Enzyme Assay

Compounds were dissolved in DMSO and preincubated with human recombinant C-terminal His-tagged LDHA (0.070 μg/mL) for 10 min at room temperature in assay buffer consisting of 50 mM Tris (pH 7.5) and 100 mM NaCl in black walled, clear bottom, non-binding 96-well plates. Equal volumes of substrate solution containing 100 μM of pyruvate and 100 μM of NADH in assay buffer was added to each well (final concentration 0.035 μg/mL enzyme, 50 μM pyruvate, 50 μM NADH, and 1% DMSO). For human serum albumin (HSA) shift assay, the compounds were preincubated with the enzyme in assay buffer containing 20% HSA before substrate addition (final concentration 10% HSA). The reaction was monitored at 340 nm on a plate reader (Molecular Devices) in kinetic mode for 15 min. The rate of the reaction was determined by plotting absorbance vs time.

Table 2 shows LDHA enzyme inhibition for the compounds described below, categorized according to potency: IC₅₀<1 nM=+++; IC₅₀ between 1 to 10 nM=++; IC₅₀ between 10 nM and 100 nM=+.

TABLE 2

| Compound | LDHA Enzyme IC₅₀ | LDHA Enzyme + HSA IC₅₀ |
|---|---|---|
| Example 1 | +++ | ++ |
| Example 2 | +++ | +++ |
| Example 3 | +++ | ++ |
| Example 4 | +++ | ++ |
| Example 5 | +++ | − |
| Example 6 | +++ | ++ |
| Example 7 | +++ | ++ |
| Example 8 | +++ | ++ |
| Example 9 | +++ | ++ |
| Example 10 | +++ | ++ |

### Example 12: Primary Mouse Hepatocyte Assay

Compounds were evaluated in an ex vivo assay consisting of fresh primary mouse hepatocytes. Hepatocytes were isolated from wild type mice (C57BL/6NCrl from Charles River Labs) using a two-step collagenase perfusion technique which involves a sequential perfusion of anesthetized mice with Hanks' balanced salt solution and collagenase. Following isolation, viable wild type hepatocytes are incubated with test compound in presence of pyruvate and compound potency is evaluated by measuring the lactate produced by the cells using liquid chromatography coupled to mass spectrometry. Chromatographic separation was achieved on an XDB-C18 4.6×50 mm column (Agilent, Cat #927975-902) at a flow rate of 1 mL/min. Mobile phase A consisted of 0.1% formic acid in water and mobile phase B consisted of acetonitrile. A gradient program was initiated starting at 5% B which was held for 1 minutes, then ramped from 5% to 95% B over 1 minute. After holding at 95% B for 1 minute, the program changed back to 5% B. The column was equilibrated with 5% B for 1.5 minutes before the next injection. The mass spectrometer was operated in negative mode with electrospray ionization. The following transition along with their collision energy (CE) were used: 89→43 (CE: −16 V) for lactate and 92→45 (CE: −16 V) for ¹³C₃-Lactate (internal standard).

Table 3 shows inhibition of lactate production in the primary mouse hepatocyte assay for the compounds described below. IC₅₀<100 nM=+++; IC₅₀ between 100 and 250 nM=++; IC₅₀ between 250 nM and 1000 nM=+.

TABLE 3

| | Primary Mouse Hepatocyte |
|---|---|
| Example | Lactate IC₅₀ |
| Example 1 | +++ |
| Example 2 | +++ |
| Example 3 | +++ |
| Example 4 | +++ |
| Example 6 | +++ |
| Example 7 | +++ |
| Example 8 | +++ |
| Example 9 | +++ |
| Example 10 | +++ |

### Example 13: PH1 AGXT Knockdown Mouse Model

To assess the in vivo efficacy of LDH inhibitors, a mouse model with hepatic knockdown of the AGXT gene was developed. The model was generated through systemic administration of 0.4 mg/kg siRNA to c57bl/6 male mice (8-12 weeks of age, Charles River Labs). The AGXT siRNA was encapsulated in a lipid nanoparticle (XL-10 (KL-52) LNP as described in WO2016/205410) and its sequence was: 5′-AcAAcuGGAGGGAcAucGudTsdT-3′ (modified sense strand sequence, N: RNA residues; dN: DNA residues; n: 2′-O-methyl residues; s: phosphorothioate residues) and 5′-ACGAUGUCCCUCcAGUUGUdTsdT-3′ (modified antisense strand sequence, see annotation above for residue modifications). Administration of the AGXT siRNA was done intravenously on day 0 and day 7 to maintain >90% knockdown of hepatic AGXT expression throughout the experimental study. The AGXT-KD model presented robust elevation of the urinary oxalate excretion within 7 days post-administration to a similar extent as AGXT-null mice (Salido, Proc Natl Acad Sci, 2006, 103 (48), 18249-18254). Prior to initiation of treatment with LDH inhibitor, oxalate and creatinine levels in urine were assessed and animals were assigned to treatment groups.

Certain select compounds disclosed herein were administered at 5 mg/kg QD (PO) per os over 5 consecutive days, starting 8 days after initial AGXT-siRNA administration. Once oral treatment was completed, mice were placed in metabolic cages and urine was collected over 24 hours. Sacrifice was performed after completion of the urine collection, and plasma/selected organs were collected and analyzed for drug concentrations.

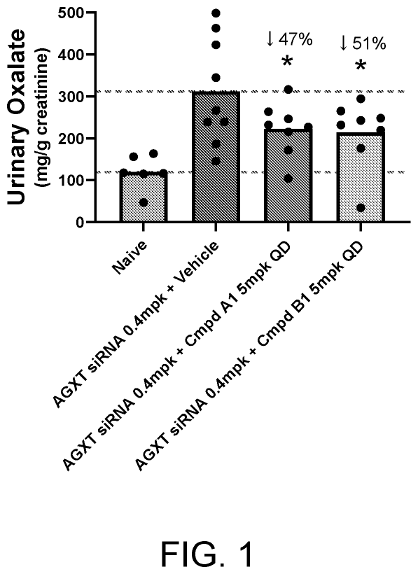

Urinary oxalate and creatinine were quantified using commercially available kits according to manufacturer's protocol (Trinity Biotech USA Inc, catalog #591; R&D Systems, Inc., catalog #KGE005). Oxalate results were normalized to creatinine to account for urine diluteness. The tested compounds disclosed herein were found to reduce urinary oxalate (normalized for creatinine) on day 5, as shown in Table 4 and in FIG. 1.

TABLE 4

| | | % Urinary Oxalate |
|---|---|---|
| | Daily Dose (mg/kg, | Reduction on day 5 |
| Compound | PO) over 5 days | (mg/g creatinine) |
| Compound A1 | 5 mg/kg QD | 47% |
| Compound B1 | 5 mg/kg QD | 51% |

### Example 14: Liver-Targeted Tissue Distribution Profile Studies

Plasma and liver (target organ) concentrations of test compounds were determined in rats following a single per os (PO) administration of the test compounds. Male Sprague Dawley rats (6-8 weeks of age, Charles River Labs) were fasted overnight and a single dose of test compounds (10 mg/kg; as a suspension in 0.5% methyl cellulose) was administered by PO gavage. Rats were then sacrificed at 4 and 24 h after administration of the test compounds, and plasma and liver tissue samples were collected. Liver tissue samples were homogenized in 5 volumes of water:acetonitrile (50/50; v/v) mixture using a bead mill homogenizer. Plasma and liver homogenate samples were processed by protein precipitation using acetonitrile and the concentrations of the test compounds were determined by liquid chromatography-tandem mass spectrometry (SciEx triple quad 5500+ with Exion UPLC).

Liver and plasma exposures at the 4 h and 24 h timepoints for the various compounds are described in Table 5 below. Measured compound concentrations are as follows: ++++>5 μM; 5 μM≥+++>1 μM; 1 μM≥++>0.25 μM; 0.25 μM≥+>0 μM.

TABLE 5

| | Measured Compound Concentrations | | | |
|---|---|---|---|---|
| | (μM) after PO dosing at 10 mg/kg to SD rats | | | |
| Compound | Liver-4 h | Plasma-4 h | Liver-24 h | Plasma-24 h |
| Example 1 | +++ | + | ++ | + |
| Example 2 | +++ | + | +++ | + |
| Example 6 | ++++ | + | +++ | + |
| Example 7 | ++++ | + | +++ | + |
| Example 9 | +++ | + | ++ | + |

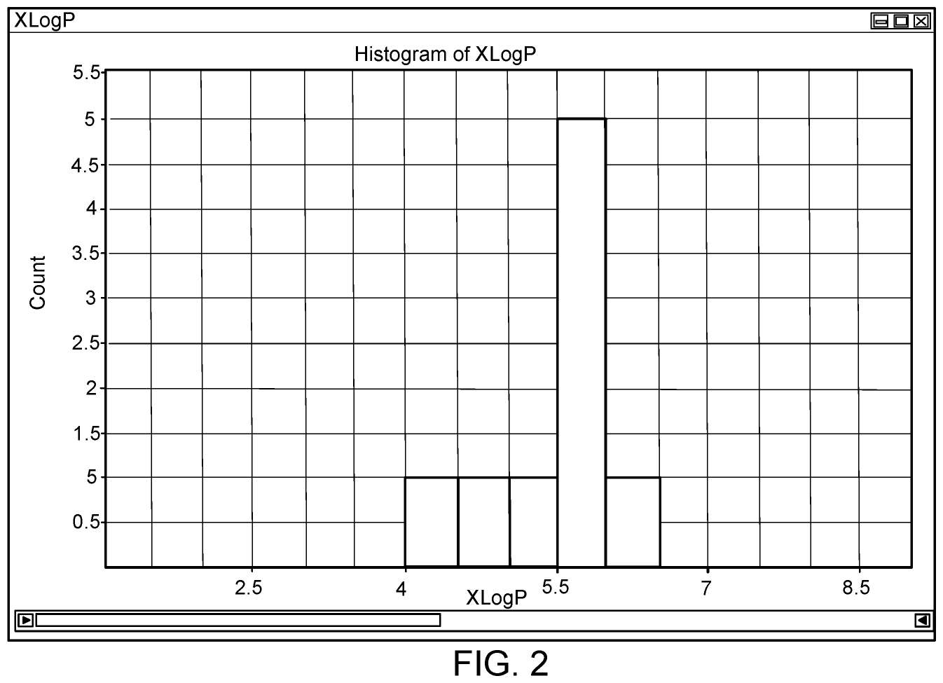

The XlogP values were also calculated for the compounds in Table 4 using Vortex v2018.09.76561.64-s, by Dotmatics Ltd. The XlogP distribution of the compounds in Table 5 are shown in FIG. 2.

### Example 15: Rat Tolerability Assessment

To assess the potential tolerability profile of the compounds provided herein, an in-house 7-day dose-limiting toxicity (DLT) studies in rats was performed. The primary variables assessed were body weight change, CBC parameters (including red-blood cells, hemoglobin, hematocrit, mean corpuscular volume, mean corpuscular hemoglobin and reticulocytes), and serum biochemistry (alanine aminotransferase, alkaline phosphatase, aspartate aminotransferase, total bilirubin and creatine kinase).

Male Sprague Dawley Rats (Charles River Laboratories) of 8 weeks of age were weighed daily and test compound was dosed per os QD over 7 consecutive days. Animals were sacrificed 24 h post 7^{th} dose.

At study termination, spleen weight was recorded, and portions of liver, muscle, testes, spleen and kidney were collected to evaluate drug levels in those organs. Blood was collected and divided as follows: 1) serum sample for biochemistry, 2) CBC+manual reticulocyte count (K₃EDTA), 3) CBC+automated reticulocyte count (K₃EDTA), and 4) plasma sample for systemic drug level assessment.

A compound disclosed herein was tested at doses of 30, 100 and 300 mg/kg PO QD, and compared to a reference LDH inhibitor disclosed as Compound ID 408 in WO2016/109559 dosed at 65 and 100 mg/kg PO QD. As shown in Table 6, the measures for body weight, spleen weight, CBC parameters and serum biochemistry after seven days were found to be in the normal range at all three doses, compared to the reference LDH inhibitor, indicating that a seven-day QD dose at 300 mg/kg of this compound disclosed herein is well tolerated in rats and provided a wider therapeutic window versus the LDH reference inhibitor (>40×).

TABLE 6

| | | | |
|---|---|---|---|
| | | | Serum |
| Body | Spleen | CBC | Bio- |
| Weight | Weight | Parameters | chemistry |

Reference Compound ID 408

| | | | | |
|---|---|---|---|---|
| 65 mg/kg, | normal range | normal range | normal range | normal range |
| PO | | | | |
| 100 mg/kg, | altered | increased | altered | altered |
| PO | | | | |

Test Compound

| | | | | |
|---|---|---|---|---|
| 30 mg/kg, | normal range | normal range | normal range | normal range |
| PO | | | | |
| 100 mg/kg, | normal range | normal range | normal range | normal range |
| PO | | | | |
| 300 mg/kg, | normal range | normal range | normal range | normal range |
| PO | | | | |

Analysis of drug concentrations of the test compound in liver, muscle, testes, spleen, kidney and plasma collected at study termination (24 h post last PO dose) showed higher liver-to-non-liver tissue ratios than was found for the reference LDH inhibitor, which suggest that this compound has greatly improved liver exposure compared to the reference compound. The reference LHD inhibitor exhibited a liver-to-muscle ratio of about 2:1 and in certain embodiments, the compound disclosed herein exhibits a liver-to-muscle ratio of greater than about 20:1, greater than about 25:1, greater than about 50:1, greater than about 80:1, or greater than about 100:1. The reference LDH inhibitor exhibited a liver-to-testes ratio of 6:1 while in certain embodiments, the compound disclosed herein exhibits a liver-to-testes ratio of greater than about 20:1, greater than about 25:1, greater than about 50:1, greater than about 80:1, or greater than about 100:1. The embodiments described above are intended to be merely exemplary, and those skilled in the art will recognize, or will be able to ascertain using no more than routine experimentation, numerous equivalents of specific compounds, materials and procedures. All such equivalents are considered to be within the scope of the claimed subject matter and are encompassed by the appended claims. Since modifications will be apparent to those of skill in the art, it is intended that the claimed subject matter be limited only by the scope of the appended claims.

What is claimed is:

1. A compound of Formula (I): 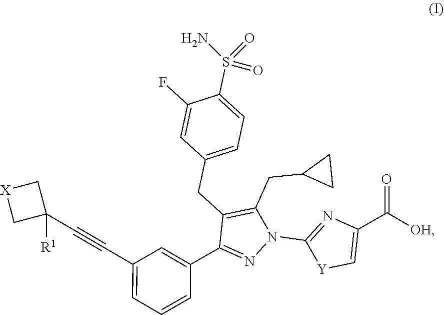
or a pharmaceutically acceptable salt or solvate thereof,
wherein X is —O— or —CH₂—;
Y is —O— or —S—; and
R¹ is hydrogen, hydroxyl, methyl, or CF₃.

2. The compound of claim 1, wherein Y is —S—.

3. The compound of claim 1, wherein X is —O—.

4. The compound of any one of claims 1 to 3, wherein R¹ is hydrogen, hydroxyl or methyl.

5. The compound of any one of claims 1 to 4, selected from the group consisting of: 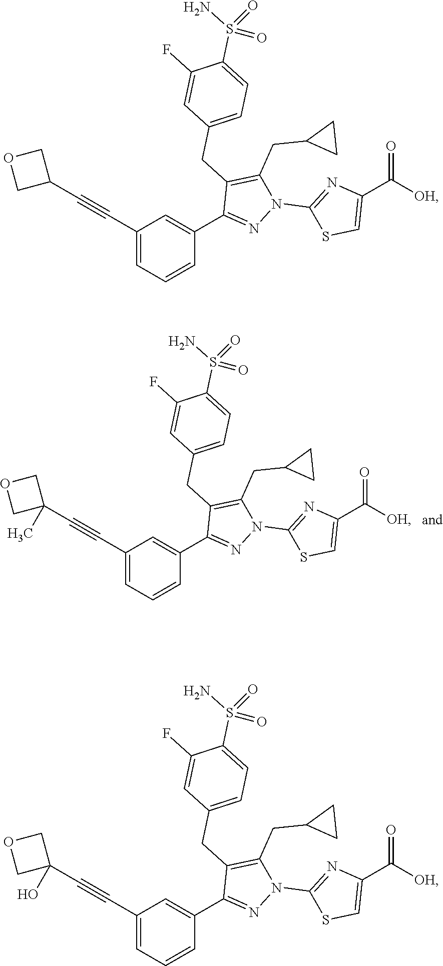 [SMILES: NS(=O)(=O)c1ccc(Cc2c(-c3cccc(C#CC4COC4)c3)nn(-c3nc(C(=O)O)cs3)c2CC2CC2)cc1F ; CC1(C#Cc2cccc(-c3nn(-c4nc(C(=O)O)cs4)c(CC4CC4)c3Cc3ccc(S(N)(=O)=O)c(F)c3)c2)COC1 ; "and" ; NS(=O)(=O)c1ccc(Cc2c(-c3cccc(C#CC4(O)COC4)c3)nn(-c3nc(C(=O)O)cs3)c2CC2CC2)cc1F]
or a pharmaceutically acceptable salt or solvate thereof.

6. A compound selected from the group consisting of: 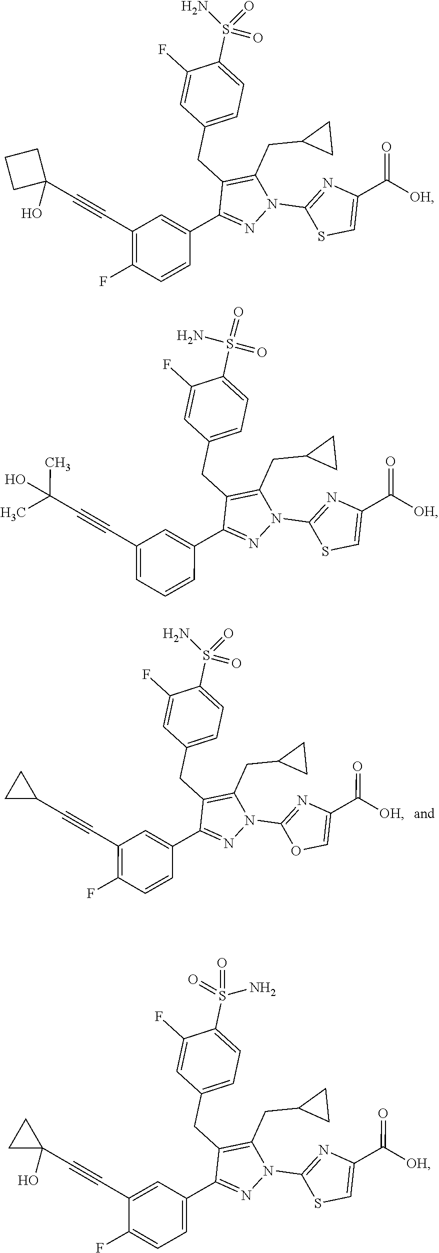
or a pharmaceutically acceptable salt or solvate thereof.

7. The compound of any one of claims 1 to 6, wherein the compound is present in the form of a pharmaceutically acceptable salt.

8. The compound of any one of claims 1 to 7, wherein the compound is present in the form of a solvate.

9. The compound of claim 8, wherein the solvate is a hydrate.

10. A pharmaceutical composition comprising a compound of any one of claims 1 to 9, and a pharmaceutically acceptable carrier.

11. A method of treating a disease or disorder associated with elevated oxalate levels, comprising administering to a subject having such disease or disorder, a therapeutically effective amount of a compound of any one of claims 1 to 9, or a pharmaceutically acceptable salt or solvate thereof, or a pharmaceutical composition of claim 10.

12. The method of claim 11, wherein the elevated oxalate levels is elevated urinary oxalate levels.

13. The method of claim 11, wherein the elevated oxalate levels is elevated plasma oxalate levels.

14. The method of any one of claims 11 to 13, wherein the disease or disorder is hyperoxaluria, chronic kidney disease (CKD), end stage renal disease (ESRD) or kidney stone disease.

15. The method of claim 14, wherein hyperoxaluria is primary hyperoxaluria or secondary hyperoxaluria.

16. The method of claim 15, wherein the primary hyperoxaluria is primary hyperoxaluria type 1 (PH-1), primary hyperoxaluria type 2 (PH-2) or primary hyperoxaluria type 3 (PH-3).

17. The method of any one or claims 11 to 16, wherein the subject with the disease or disorder has an AGXT, GRHPR or HOGA1 mutation, or a combination of mutations thereof.

18. A method of lowering oxalate levels in a subject in need thereof, comprising administering to the subject a therapeutically effective amount of a compound of any one of claims 1 to 9, or a pharmaceutically acceptable salt or solvate thereof, or a pharmaceutical composition of claim 10.

19. A method of treating kidney stone formation in a subject in need thereof comprising administering to the subject a therapeutically effective amount of a compound of any one of claims 1 to 9, or a pharmaceutically acceptable salt or solvate thereof, or a pharmaceutical composition of claim 10.

20. The method of any one of claims 11 to 19, further comprising administering to the subject a therapeutically effective amount of a second therapeutic agent.

21. The method of claim 20, wherein the second therapeutic agent is a glyoxylate- or oxalate-lowering therapeutic.

22. The method of 21, wherein the glyoxylate- or oxalate-lowering therapeutic is an RNAi therapeutic.

23. The method of claim 22, wherein the glyoxylate- or oxalate-lowering therapeutic is lumasiran, nedosiran, reloxaliase, stiripentol, oxalobacter formigenes or vitamin B6.

24. The compound of any one of claims 1 to 9, or a pharmaceutically acceptable salt or solvate thereof, or a pharmaceutical composition of claim 10, for use in treating a disease or disorder associated with elevated oxalate levels.

25. The compound or pharmaceutical composition for use in claim 24, wherein the disease or disorder is hyperoxaluria, chronic kidney disease (CKD), end stage renal disease (ESRD) or kidney stone disease.

26. The compound or pharmaceutical composition for use in claim 24, wherein the hyperoxaluria is primary hyperoxaluria or secondary hyperoxaluria.

27. The compound or pharmaceutical composition for use in claim 26, wherein the primary hyperoxaluria is primary hyperoxaluria type 1 (PH-1), primary hyperoxaluria type 2 (PH-2) or primary hyperoxaluria type 3 (PH-3).

28. The compound or pharmaceutical composition for use in any one of claims 24 to 27, wherein the disease or disorder is associated with an AGXT, GRHPR or HOGA1 mutation, or a combination of mutations thereof.

29. A method of treating a disease or disorder associated with elevated oxalate levels, comprising administering to a subject having such disease or disorder, a therapeutically effective amount of a compound selected from the group consisting of: 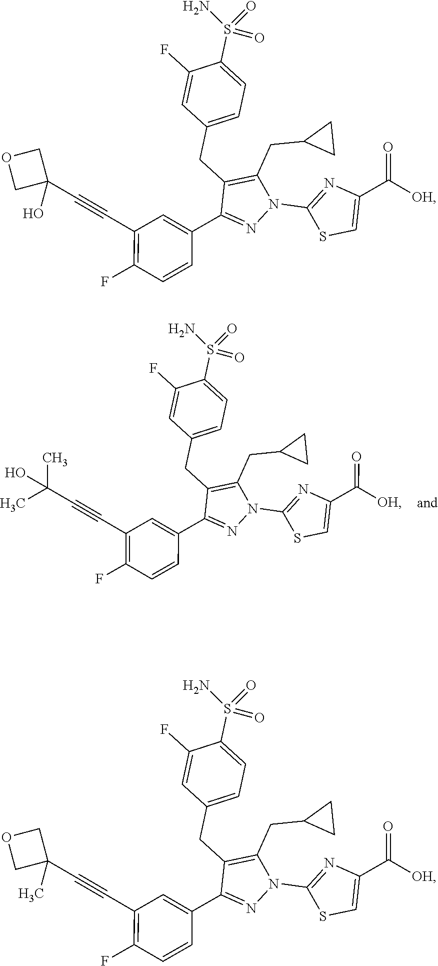 [SMILES: NS(=O)(=O)c1ccc(Cc2c(-c3ccc(F)c(C#CC4(O)COC4)c3)nn(-c3nc(C(=O)O)cs3)c2CC2CC2)cc1F ; CC(C)(O)C#Cc1cc(-c2nn(-c3nc(C(=O)O)cs3)c(CC3CC3)c2Cc2ccc(S(N)(=O)=O)c(F)c2)ccc1F ; "and" ; CC1(C#Cc2cc(-c3nn(-c4nc(C(=O)O)cs4)c(CC4CC4)c3Cc3ccc(S(N)(=O)=O)c(F)c3)ccc2F)COC1]
or a pharmaceutically acceptable salt or solvate thereof.

30. The method of claim 29, wherein the elevated oxalate levels is elevated urinary oxalate levels.

31. The method of claim 29, wherein the elevated oxalate levels is elevated plasma oxalate levels.

32. The method of claim 31, wherein the disease or disorder is hyperoxaluria, chronic kidney disease (CKD), end stage renal disease (ESRD) or kidney stone disease.

33. The method of claim 32, wherein the hyperoxaluria is primary hyperoxaluria or secondary hyperoxaluria.

34. The method of claim 33, wherein the primary hyperoxaluria is primary hyperoxaluria type 1 (PH-1), primary hyperoxaluria type 2 (PH-2) or primary hyperoxaluria type 3 (PH-3).

35. The method of any one or claims 29 to 34, wherein the subject with the disease or disorder has an AGXT, GRHPR or HOGA1 mutation, or a combination of mutations thereof.

36. A method of lowering oxalate levels in a subject in need thereof, comprising administering to the subject a compound selected from the group consisting of: 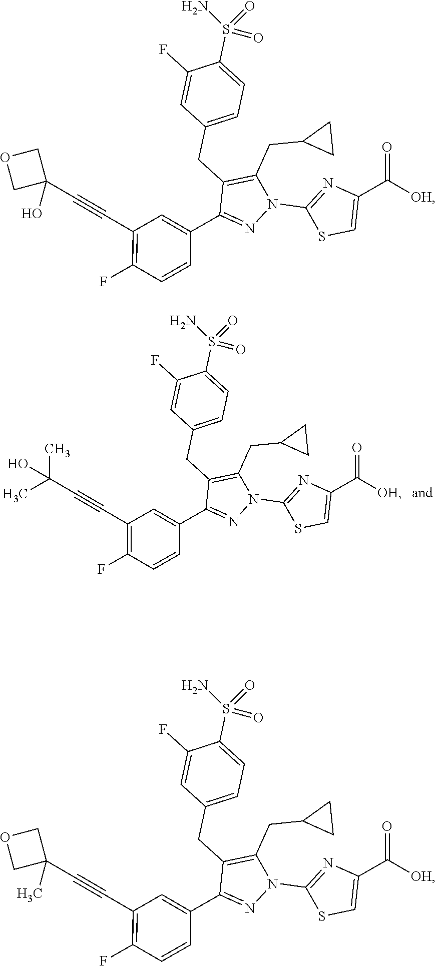 [SMILES: NS(=O)(=O)c1ccc(Cc2c(-c3ccc(F)c(C#CC4(O)COC4)c3)nn(-c3nc(C(=O)O)cs3)c2CC2CC2)cc1F ; CC(C)(O)C#Cc1cc(-c2nn(-c3nc(C(=O)O)cs3)c(CC3CC3)c2Cc2ccc(S(N)(=O)=O)c(F)c2)ccc1F ; "and" ; CC1(C#Cc2cc(-c3nn(-c4nc(C(=O)O)cs4)c(CC4CC4)c3Cc3ccc(S(N)(=O)=O)c(F)c3)ccc2F)COC1]
or a pharmaceutically acceptable salt or solvate thereof.

37. The method of treating kidney stone formation in a subject in need thereof comprising administering to the subject a compound selected from the group consisting of: 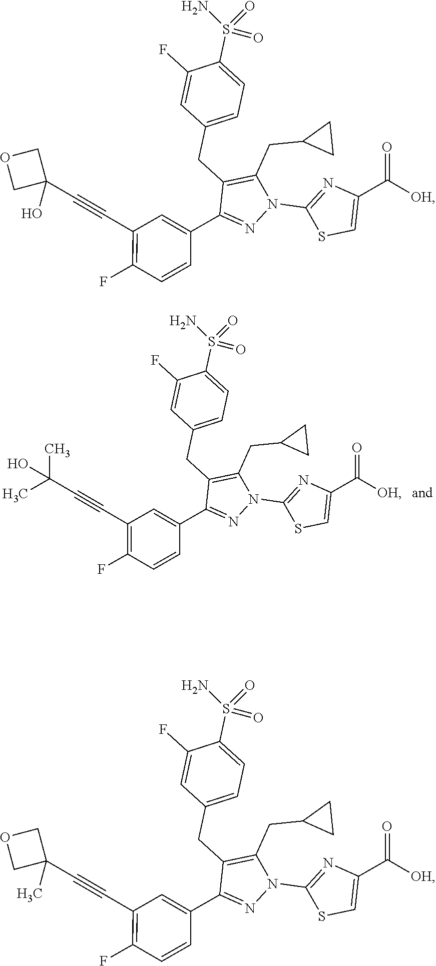 [SMILES: NS(=O)(=O)c1ccc(Cc2c(-c3ccc(F)c(C#CC4(O)COC4)c3)nn(-c3nc(C(=O)O)cs3)c2CC2CC2)cc1F ; CC(C)(O)C#Cc1cc(-c2nn(-c3nc(C(=O)O)cs3)c(CC3CC3)c2Cc2ccc(S(N)(=O)=O)c(F)c2)ccc1F ; "and" ; CC1(C#Cc2cc(-c3nn(-c4nc(C(=O)O)cs4)c(CC4CC4)c3Cc3ccc(S(N)(=O)=O)c(F)c3)ccc2F)COC1]
or a pharmaceutically acceptable salt or solvate thereof.

38. The compound selected from the group consisting of: 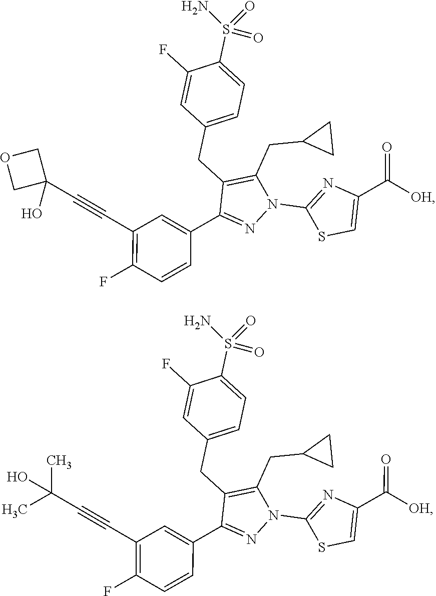 [SMILES: NS(=O)(=O)c1ccc(Cc2c(-c3ccc(F)c(C#CC4(O)COC4)c3)nn(-c3nc(C(=O)O)cs3)c2CC2CC2)cc1F ; CC(C)(O)C#Cc1cc(-c2nn(-c3nc(C(=O)O)cs3)c(CC3CC3)c2Cc2ccc(S(N)(=O)=O)c(F)c2)ccc1F]
or a pharmaceutically acceptable salt or solvate thereof, for use in treating a disease or disorder associated with elevated oxalate levels.

39. The compound for use in claim 38, wherein the disease or disorder is hyperoxaluria, chronic kidney disease (CKD), end stage renal disease (ESRD) or kidney stone disease.

40. The compound for use in claim 38 or 39, wherein the hyperoxaluria is primary hyperoxaluria or secondary hyperoxaluria.

41. The compound for use in claim 40, wherein the primary hyperoxaluria is primary hyperoxaluria type 1 (PH-1), primary hyperoxaluria type 2 (PH-2) or primary hyperoxaluria type 3 (PH-3).

42. The compound for use in claim 38, wherein the disease or disorder is associated with an AGXT, GRHPR or HOGA1 mutation, or a combination of mutations thereof.
